(19) United States
(12) Patent Application Publication
(10) Pub. No.: US 2026/0147141 A1
(43) Pub. Date: May 28, 2026

(54) SURFACE MODIFIED SILICONE OPHTHALMIC DEVICES

(71) Applicant: Bausch + Lomb Ireland Limited, Dublin (IE)

(72) Inventors: Feng-Yang Shih, North Chili, NY (US); Sankarsan Biswas, Rochester, NY (US); Anna Flach, North Chili, NY (US); Jacob Cole, Rochester, NY (US); Alok Kumar Awasthi, Pittsford, NY (US)

(21) Appl. No.: 19/344,947

(22) Filed: Sep. 30, 2025

Related U.S. Application Data
(60) Provisional application No. 63/724,988, filed on Nov. 26, 2024.

(51) Int. Cl. G02B 1/18; A61L 12/04; A61L 12/08; C08J 7/04; C08J 7/056; G02B 1/04

(52) U.S. Cl. CPC G02B 1/18; A61L 12/04; A61L 12/086; C08J 7/0427; C08J 7/056; G02B 1/043; C08J 2333/26; C08J 2383/04

(57) ABSTRACT

A surface modified ophthalmic device includes a silicone ophthalmic device having a hydrophilic surface coating attached to a surface of the silicone ophthalmic device by van der Waals dispersion forces, the hydrophilic surface coating comprising a copolymer comprising (i) monomeric units derived from an ethylenically unsaturated-containing monomer having ring-opening reactive functionalities, and (ii) monomeric units derived from a hydrophilic monomer having an ethylenically unsaturated reactive group.

### PRIORITY CLAIM

The present application claims priority to U.S. Provisional Patent Application Ser. No. 63/724,988, entitled “Surface Modified Silicone Ophthalmic Devices,” filed Nov. 26, 2024, the content of which is incorporated by reference herein in its entirety.

### BACKGROUND

Ophthalmic devices such as contact lenses made from, for example, silicone-containing materials, have been investigated for a number of years. Such materials can generally be subdivided into two major classes, namely hydrogels and non-hydrogels. Hydrogels can absorb and retain water in an equilibrium state, whereas non-hydrogels do not absorb appreciable amounts of water. Regardless of their water content, both hydrogel and non-hydrogel silicone medical devices tend to have relatively hydrophobic, non-wettable surfaces that have a high affinity for lipids. This problem is of particular concern with contact lenses.

Those skilled in the art have long recognized the need for modifying the surface of the silicone ophthalmic devices such as silicone contact lenses so that they are compatible with the eye. For example, by increasing the hydrophilicity of a contact lens surface, the wettability of the contact lens can be improved. This, in turn, is associated with improved wear comfort of the contact lenses. Additionally, the surface of the lens can affect the lens's susceptibility to deposition, particularly the deposition of proteins and lipids resulting from tear fluid during lens wear. Accumulated deposition can cause eye discomfort or even inflammation. In the case of extended wear lenses (i.e., lenses used without daily removal of the lens before sleep), the surface is especially important, since extended wear lenses must be designed for high standards of comfort and biocompatibility over an extended period of time.

### SUMMARY

In accordance with an illustrative embodiment, a surface modified silicone ophthalmic device comprises a silicone ophthalmic device having a hydrophilic surface coating attached to a surface of the silicone ophthalmic device by van der Waals dispersion forces, the hydrophilic surface coating comprising a copolymer comprising (i) monomeric units derived from an ethylenically unsaturated-containing monomer having ring-opening reactive functionalities, and (ii) monomeric units derived from a hydrophilic monomer having an ethylenically unsaturated reactive group.

In accordance with another illustrative embodiment, a method for making a surface modified silicone ophthalmic device comprises attaching a hydrophilic surface coating to a surface of a silicone ophthalmic device by van der Waals dispersion forces, the hydrophilic surface coating comprising a copolymer comprising (i) monomeric units derived from an ethylenically unsaturated-containing monomer having ring-opening reactive functionalities, and (ii) monomeric units derived from a hydrophilic monomer having an ethylenically unsaturated reactive group.

### DETAILED DESCRIPTION

Various illustrative embodiments described herein include silicone ophthalmic devices having a hydrophilic surface coating attached to a surface of the silicone ophthalmic device by van der Waals dispersion forces.

### Definitions

To define more clearly the terms used herein, the following definitions are provided. Unless otherwise indicated, the following definitions are applicable to this disclosure. If a term is used in this disclosure but is not specifically defined herein, the definition from the IUPAC Compendium of Chemical Terminology can be applied, as long as that definition does not conflict with any other disclosure or definition applied herein or render indefinite or non-enabled any claim to which that definition is applied. To the extent that any definition or usage provided by any document incorporated herein by reference conflicts with the definition or usage provided herein, the definition or usage provided herein controls.

A “prepolymer” refers to a starting polymer which can be cured (e.g., crosslinked and/or polymerized) actinically or thermally or chemically to obtain a crosslinked and/or polymerized polymer having a molecular weight higher than the molecular weight of the starting polymer.

A “crosslinkable prepolymer” refers to a starting polymer which can be crosslinked to obtain a crosslinked polymer having a molecular weight higher than the molecular weight of the starting polymer.

As used herein, the term “SiHy” shall be understood to mean silicone hydrogel.

As used herein, the term “hydrogel” or “hydrogel material” refers to a crosslinked polymeric material that has three-dimensional polymer networks (i.e., polymer matrix), is insoluble in water, but can hold at least 10 percent by weight of water in its polymer matrix when it is fully hydrated.

As used herein, the term “silicone hydrogel” or “SiHy” interchangeably refers to a hydrogel containing silicone. A silicone hydrogel (SiHy) typically is obtained by copolymerization of a polymerizable composition comprising at least one silicone-containing vinylic monomer or at least one silicone-containing vinylic macromer or at least one silicone-containing prepolymer having ethylenically unsaturated groups.

As used herein, the term “ophthalmic device” refers to ophthalmic devices that reside in or on the eye. These devices can provide optical correction, wound care, drug delivery, diagnostic functionality or cosmetic enhancement or effect or a combination of these properties. Suitable ophthalmic devices include, for example, ophthalmic lenses such as soft contact lenses, e.g., a soft, hydrogel lens; soft, non-hydrogel lens and the like, hard contact lenses, e.g., a hard, gas permeable lens material and the like, intraocular lenses, overlay lenses, ocular inserts, optical inserts and the like. A contact lens can be in a dry state or a wet state. A “dry state” refers to a soft contact lens in a state prior to hydration or the state of a hard lens under storage or use conditions. A “wet state” refers to a soft contact lens in a hydrated state. As is understood by one skilled in the art, a contact lens is considered to be “soft” if it can be folded back upon itself without breaking. A contact lens can be tinted before printing any color patterns.

The term “(meth)” as used herein denotes an optional methyl substituent. Thus, terms such as “(meth)acrylate” denotes either methacrylate or acrylate, and “(meth)acrylamide” denotes either methacrylamide or acrylamide.

While compositions and processes are described in terms of “comprising” various components or steps, the compositions and processes can also “consist essentially of” or “consist of” the various components or steps, unless stated otherwise.

The terms “a,” “an,” and “the” are intended to include plural alternatives, e.g., at least one. The terms “including,” “with,” and “having,” as used herein, are defined as comprising (i.e., open language), unless specified otherwise.

Various numerical ranges are disclosed herein. When Applicant discloses or claims a range of any type, Applicant's intent is to disclose or claim individually each possible number that such a range could reasonably encompass, including end points of the range as well as any sub-ranges and combinations of sub-ranges encompassed therein, unless otherwise specified. For example, all numerical end points of ranges disclosed herein are approximate, unless excluded by proviso.

Values or ranges may be expressed herein as “about,” from “about” one particular value, and/or to “about” another particular value. When such values or ranges are expressed, other embodiments disclosed include the specific value recited, from the one particular value, and/or to the other particular value. Similarly, when values are expressed as approximations, by use of the antecedent “about,” it will be understood that the particular value forms another embodiment. It will be further understood that there are a number of values disclosed therein, and that each value is also herein disclosed as “about” that particular value in addition to the value itself. In another aspect, use of the term “about” means ±20% of the stated value, ±15% of the stated value, ±10% of the stated value, ±5% of the stated value, ±3% of the stated value, or ±1% of the stated value.

The terms “wt. %,” “vol. %” or “mol. %” refers to a weight, volume, or molar percentage of a component, respectively, based on the total weight, the total volume, or the total moles of material that includes the component. In a non-limiting example, 10 moles of component in 100 moles of the material are 10 mol. % of component.

Applicant reserves the right to proviso out or exclude any individual members of any such group of values or ranges, including any sub-ranges or combinations of sub-ranges within the group, that can be claimed according to a range or in any similar manner, if for any reason Applicant chooses to claim less than the full measure of the disclosure, for example, to account for a reference that Applicant may be unaware of at the time of the filing of the application. Further, Applicant reserves the right to proviso out or exclude any members of a claimed group.

Silicone hydrogels (SiHy) such as contact lenses, which are made of a hydrated, crosslinked polymeric material that contains silicone and a certain amount of water within the lens polymer matrix at equilibrium, are increasingly becoming popular as compared to the conventional hydrogels. In the field of contact lenses, various physical and chemical properties such as, for example, oxygen permeability, wettability, material strength and stability, are but a few of the factors that must be carefully balanced in order to provide a useable contact lens. For example, oxygen permeability is a desirable property for contact lens materials since the human cornea will be damaged if it is deprived of oxygen for an extended period. Oxygen permeability is conventionally expressed in units of Barrer, and also called Dk. Oxygen transmissibility is a property of contact lens materials related to oxygen permeability where oxygen permeability is divided by lens thickness, or Dk/t. Wettability also is important in that, if the lens is not sufficiently wettable, it does not remain lubricated and therefore cannot be worn comfortably in the eye.

As mentioned above, by increasing the hydrophilicity of a silicone ophthalmic device such as a silicone contact lens surface, the wettability of the silicone contact lens can be improved. This, in turn, is associated with improved wear comfort of the contact lenses. Additionally, the surface of the lens can affect the lens's susceptibility to deposition, particularly the deposition of proteins and lipids resulting from tear fluid during lens wear. Accumulated deposition can cause eye discomfort or even inflammation. In the case of extended wear lenses (i.e., lenses used without daily removal of the lens before sleep), the surface is especially important, since extended wear lenses must be designed for high standards of comfort and biocompatibility over an extended period of time.

Previous attempts at forming a surface modified contact lens used a surface coating derived from a copolymer of glycidyl methacrylate and dimethylacrylamide. The surface coating derived from a copolymer of glycidyl methacrylate and dimethylacrylamide was attached to the contact lens by covalent bonding of the epoxide moiety of the glycidyl methacrylate with surface reactive functional groups of the contact lens. A problem associated with this surface coating is that the surface modified contact lens failed to achieve the robustness necessary for use of the contact lens in the human eye for an extended period of time. For example, while the inner surface of the contact lens was sufficiently coated with the surface coating, the outer surface of the contact lens had a relatively thinner layer of the surface coating relative to the layer of the inner surface. Accordingly, it would be desirable to provide improved surface modified silicone ophthalmic devices having a highly wettable and/or lubricious surface coating that is uniform on the inner surface and the outer surface while also exhibiting the robustness necessary for long term use of the silicone ophthalmic device.

The non-limiting illustrative embodiments disclosed herein overcome the foregoing drawbacks by providing a surface modified silicone ophthalmic device comprising a silicone ophthalmic device having a hydrophilic surface coating attached to a surface of the silicone ophthalmic device by van der Waals dispersion forces, the hydrophilic surface coating comprising a copolymer comprising (i) monomeric units derived from an ethylenically unsaturated-containing monomer having ring-opening reactive functionalities, and (ii) monomeric units derived from a hydrophilic monomer having an ethylenically unsaturated reactive group.

The surface modified silicone ophthalmic devices according to the non-limiting illustrative embodiments described herein advantageously provide a robust hydrophilic surface coating on both the inner surface and the outer surface of the silicone ophthalmic device thereby allowing the use of the surface modified silicone ophthalmic device in the human eye for an extended period of time. In addition, the surface-modified silicone ophthalmic device provides a uniformity of the hydrophilic surface coating along with an increase in lubricity on both anterior and posterior sides of the silicone ophthalmic device. The uniform surface coating is advantageously formed by attaching a hydrophilic surface coating composition to a surface of a silicone ophthalmic device by van der Waals dispersion forces, the hydrophilic surface coating composition comprising a copolymer comprising (i) monomeric units derived from an ethylenically unsaturated-containing monomer having ring-opening reactive functionalities, and (ii) monomeric units derived from a hydrophilic monomer having an ethylenically unsaturated reactive group, and (b) a hydrophilic polymer having reactive functional groups.

### Silicone Ophthalmic Device

The type of silicone ophthalmic device to be coated with the hydrophilic surface coating disclosed herein is not critical and any silicone ophthalmic device is contemplated. For example, silicone hydrogel materials comprise hydrated, cross-linked silicone polymeric systems containing water in an equilibrium state. Silicone hydrogel materials contain about 5 wt. % water or more (up to, for example, about 80 wt. %).

A wide variety of materials can be used herein, and silicone hydrogel contact lens materials are particularly preferred. Silicone hydrogels generally have a water content greater than about 5 wt. % and more commonly between about 10 to about 80 wt. %. Such materials are usually prepared by polymerizing a monomeric mixture containing at least one silicone-containing monomer and at least one hydrophilic monomer. In an illustrative embodiment, the one or more silicone monomers can include, as a class of representative silicone monomers, one or more monofunctional silicone monomers represented by a structure of Formula I:

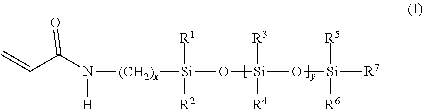

wherein R¹, R², R³ and R⁴ are independently hydrogen, an alkyl group, a halo alkyl group, a cycloalkyl group, a heterocycloalkyl group, an alkenyl group, a haloalkenyl group, an aryl group and a heteroaryl group; R⁵, R⁶ and R⁷ are independently a straight or branched alkyl group; x is from 1 to 6; and y is from 3 to 15.

In some embodiments, R¹, R², R³ and R⁴ are independently hydrogen, a C₁ to C₁₂ alkyl group, a C₁ to C₁₂ halo alkyl group, a C₃ to C₁₂ cycloalkyl group, a C₃ to C₁₂ heterocycloalkyl group, a C₂ to C₁₂ alkenyl group, a C₂ to C₁₂ haloalkenyl group, a C₆ to C₁₂ aromatic group and a C₆ to C₁₂ heteroaromatic group; R⁵, R⁶ and R⁷ are independently a straight or branched C₁ to C₁₂ alkyl group; x is from 1 to 6; and y is from 3 to 15.

In some embodiments, R¹, R², R³ and R⁴ are independently hydrogen, a C₁ to C₆ alkyl group; R⁵, R⁶ and R⁷ are independently a straight or branched C₁ to C₆ alkyl group; x is from 1 to 6; and y is from 3 to 15.

In some embodiments, R¹, R², R³ and R⁴ are independently a C₁ to C₃ alkyl group; R⁵ and R⁶ are independently a C₁ to C₃ alkyl group; R⁷ is a straight or branched C₃ to C₆ alkyl group; x is from 2 to 4; and y is from 3 to 15.

Representative examples of alkyl groups for use herein include, by way of example, a straight or branched alkyl chain radical containing carbon and hydrogen atoms of from 1 to about 30 carbon atoms or from 1 to about 12 carbon atoms or from 1 to about 6 carbon atoms with or without unsaturation, to the rest of the molecule, e.g., methyl, ethyl, n-propyl, 1-methylethyl (isopropyl), n-butyl, n-pentyl, methylene, ethylene, etc., and the like, optionally containing one or more heteroatoms, e.g., O and N, and the like, or one or more halogen atoms, e.g., fluorine, chlorine, bromine, and iodine, to form a halo alkyl group.

Representative examples of cycloalkyl groups for use herein include, by way of example, a substituted or unsubstituted, non-aromatic mono or multicyclic ring system of about 3 to about 30 carbon atoms or from 3 to about 12 carbon atoms or from 3 to about 6 carbon atoms such as, for example, cyclopropyl, cyclobutyl, cyclopentyl, cyclohexyl, perhydronapththyl, adamantyl and norbornyl groups, bridged cyclic groups or sprirobicyclic groups, e.g., spiro-(4, 4)-non-2-yl and the like, optionally containing one or more heteroatoms, e.g., O and N, and the like to form a heterocycloalkyl group.

Representative examples of cycloalkylalkyl groups for use herein include, by way of example, a substituted or unsubstituted, cyclic ring-containing radical containing from about 4 to about 30 carbon atoms or from 3 to about 6 carbon atoms directly attached to the alkyl group which are then attached to the main structure of the monomer at any carbon from the alkyl group that results in the creation of a stable structure such as, for example, cyclopropylmethyl, cyclobutylethyl, cyclopentylethyl and the like, wherein the cyclic ring can optionally contain one or more heteroatoms, e.g., O and N, and the like to form a heterocycloalkylalkyl group.

Representative examples of cycloalkenyl groups for use herein include, by way of example, a substituted or unsubstituted cyclic ring-containing radical containing from about 3 to about 30 carbon atoms or from 3 to about 6 carbon atoms with at least one carbon-carbon double bond such as, for example, cyclopropenyl, cyclobutenyl, cyclopentenyl and the like, wherein the cyclic ring can optionally contain one or more heteroatoms, e.g., O and N, and the like to form a heterocycloalkenyl group.

Representative examples of aryl groups for use herein include, by way of example, a substituted or unsubstituted, monoaromatic or polyaromatic radical containing from about 6 to about 30 carbon atoms or from about 6 to about 12 carbon atoms such as, for example, phenyl, naphthyl, tetrahydronapthyl, indenyl, biphenyl and the like, optionally containing one or more heteroatoms, e.g., O and N, and the like to form a heteroaryl group.

In an illustrative embodiment, the monofunctional silicone monomer represented by the structure of Formula I disclosed herein can be prepared according to the following reaction Scheme I.

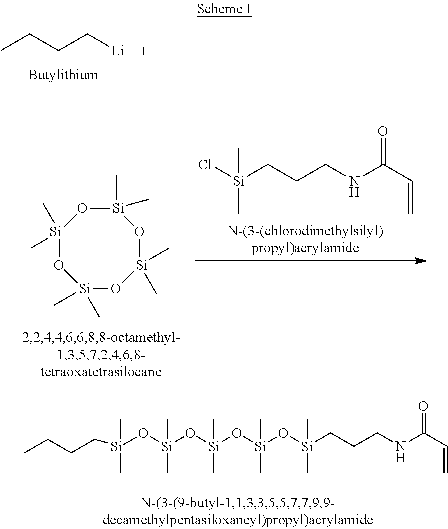

The one or more silicone monomers can further include, as a class of representative silicone monomers, one or more non-bulky organosilicon-containing monomers. An “organosilicon-containing monomer” as used herein contains at least one [siloxanyl] or at least one [silyl-alkyl-siloxanyl] repeating unit, in a monomer, macromer or prepolymer. In an illustrative embodiment, an example of a non-bulky organosilicon-containing monomer is represented by a structure of Formula IIa:

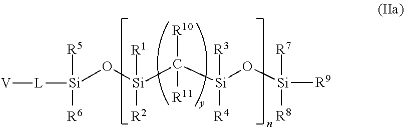

wherein V is an ethylenically unsaturated polymerizable group, L is a linking group or a bond; R¹, R², R³, R⁴, R⁵, R⁶, R⁷, R⁸, and R⁹ are independently hydrogen, an alkyl group, a haloalkyl group, a cycloalkyl group, a heterocycloalkyl group, an alkenyl group, a halo alkenyl group, or an aryl group; R¹⁰ and R¹¹ are independently hydrogen or an alkyl group wherein at least one of R¹⁰ and R¹¹ is hydrogen; y is 2 to 7 and n is 1 to 100 or from 1 to 20.

Ethylenically unsaturated polymerizable groups are well known to those skilled in the art. Suitable ethylenically unsaturated polymerizable groups include, for example, (meth)acrylates, vinyl carbonates, O-vinyl carbamates, N-vinyl carbamates, and (meth)acrylamides.

Linking groups can be any divalent radical or moiety and include, for example, substituted or unsubstituted C₁ to C₁₂ alkyl group, an alkyl ether group, an alkenyl group, an alkenyl ether group, a halo alkyl group, a substituted or unsubstituted siloxane group, and monomers capable of propagating ring opening.

In some embodiments, V is a (meth)acrylate, L is a C₁ to C₁₂ alkylene group, R¹, R², R³, R⁴, R⁵, R⁶, R⁷, R⁸, and R⁹ are independently a C₁ to C₁₂ alkyl group, R¹⁰ and R¹¹ are independently H or a C₁ to C₁₂ alkyl group, y is 2 to 7 and n is 3 to 8.

In some embodiments, V is a (meth)acrylate, L is a C₁ to C₆ alkyl group, R¹, R², R³, R⁴, R⁵, R⁶, R⁷, R⁸, and R⁹ are independently a C₁ to C₆ alkyl group, R¹⁰ and R¹¹ are independently H or a C₁ to C₆ alkyl group, y is 2 to 7 and n is 1 to 20.

Non-bulky organosilicon-containing monomers represented by a structure of Formula IIa are known in the art, see, e.g., U.S. Pat. Nos. 7,915,323, 7,994,356, 8,420,711, 8,827,447 and 9,039,174, the contents of which are incorporated by reference herein.

In an illustrative embodiment, as may be combined with one or more of the preceding paragraphs, the one or more non-bulky organosilicon-containing monomers can also comprise a monomer represented by a structure of Formula IIb:

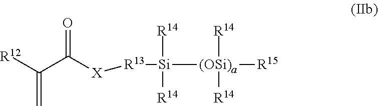

wherein R¹² is H or methyl; X is O or NR¹⁶; wherein R¹⁶ is selected from H, or C₁ to C₄ alkyl, which may be further substituted with one or more hydroxyl groups, and in some embodiments is H or methyl; R¹³ is a divalent alkyl group, which may further be functionalized with a group selected from the group consisting of ether groups, hydroxyl groups, carbamate groups and combinations thereof, and in another embodiment a C₁ to C₆ alkylene group which may be substituted with ether, hydroxyl and combinations thereof, and in yet another embodiment a C₁ or C₃ to C₄ alkylene group which may be substituted with ether, hydroxyl and combinations thereof; each R¹⁴ is independently a phenyl or a C₁ to C₄ alkyl group which may be substituted with fluorine, hydroxyl or ether, and in another embodiment each R¹⁴ is independently selected from ethyl and methyl groups, and in yet another embodiment, each R¹⁴ is methyl; R¹⁵ is a C₁ to C₄ alkyl group; a is 2 to 50, and in some embodiments 5 to 15.

Non-bulky organosilicon-containing monomers represented by a structure of Formula IIb are known in the art, see, e.g., U.S. Pat. Nos. 8,703,891, 8,937,110, 8,937,111, 9,156,934 and 9,244,197, the contents of which are incorporated by reference herein.

Representative examples of the non-bulky organosilicon-containing monomers include:
M1EDS6: a compound having the structure and available from Gelest:

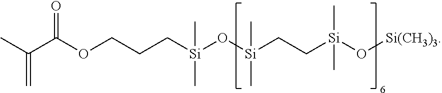

MCR-M11: a compound having the structure:

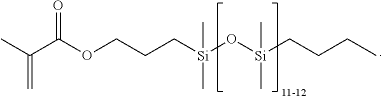

M1-MCR-C12: a compound having the structure:

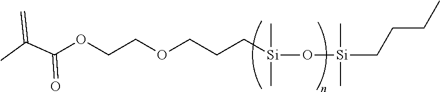

wherein n is an average of 12.

The one or more silicone monomers can further include, as a class of representative silicone monomers, one or more polysiloxane prepolymers represented by a structure of Formula III:

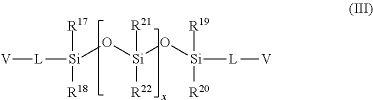

wherein each V is an independently reactive functional end group and includes, by way of example, a hydroxyl-containing reactive functional end group, and an amine-containing reactive functional end group, R¹⁷ to R²² are independently straight or branched, substituted or unsubstituted C₁-C₃₀ alkyl group, a substituted or unsubstituted C₃-C₃₀ cycloalkyl group, a substituted or unsubstituted C₄-C₃₀ cycloalkylalkyl group, a substituted or unsubstituted C₃-C₃₀ cycloalkenyl group, a substituted or unsubstituted C₆-C₃₀ aryl group, and a substituted or unsubstituted C₇-C₃₀ arylalkyl group, and L is independently a linking group.

A hydroxyl-containing reactive functional end group for use herein is a group of the general formula —OH. Representative examples of amine-containing reactive functional end groups for use herein include, by way of example, a (meth)acrylamide-containing reactive functional end group.

Linking group L is independently a straight or branched alkyl group, cycloalkyl group, an aryl group, an ether or polyether group, and an ester group as defined herein.

A representative example of a polysiloxane prepolymer is as follows:

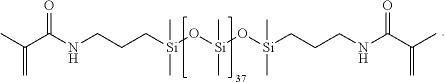

Methods for making the polysiloxane prepolymers described herein are well known and within the purview of one skilled in the art. In addition, the polysiloxane prepolymers are also commercially available from such sources as, for example, Gelest, Silar, Shin-Etsu, Momentive and Siltech.

The one or more silicone monomers can further include, as a class of representative silicone monomers, one or more silicone-containing vinyl carbonate or vinyl carbamate monomers. Suitable one or more silicone-containing vinyl carbonate or vinyl carbamate monomers include, for example, 1,3-bis[4-vinyloxycarbonyloxy)but-1-yl]tetramethyl-disiloxane; 3-(trimethylsilyl)propyl vinyl carbonate; 3-(vinyloxycarbonylthio)propyl-[tris(trimethylsiloxy)silane]; 3-[tris(trimethylsiloxy)silyl]propyl vinyl carbamate; 3-[tris(trimethylsiloxy)silyl]propyl allyl carbamate; 3-[tris(trimethylsiloxy)silyl]propyl vinyl carbonate; t-butyldimethylsiloxyethyl vinyl carbonate; trimethylsilylethyl vinyl carbonate; trimethylsilylmethyl vinyl carbonate and the like and mixtures thereof.

The one or more silicone monomers can further include, as a class of representative silicone monomers, one or more polyurethane-polysiloxane macromonomers (also sometimes referred to as prepolymers), which may have hard-soft-hard blocks like traditional urethane elastomers. They may be end-capped with a hydrophilic monomer such as HEMA. Examples of such silicone urethanes are disclosed in a variety or publications, including Lai, Yu-Chin, “The Role of Bulky Polysiloxanylalkyl Methacrylates in Polyurethane-Polysiloxane Hydrogels,” Journal of Applied Polymer Science, Vol. 60, 1193-1199 (1996). PCT Published Application No. WO 96/31792 discloses examples of such monomers, which disclosure is hereby incorporated by reference in its entirety. Further examples of silicone urethane monomers are represented by Formulae IV and V:

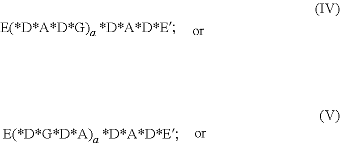

wherein:
   D independently denotes an alkyl diradical, an alkyl cycloalkyl diradical, a cycloalkyl diradical, an aryl diradical or an alkylaryl diradical having 6 to about 30 carbon atoms;
   G independently denotes an alkyl diradical, a cycloalkyl diradical, an alkyl cycloalkyl diradical, an aryl diradical or an alkylaryl diradical having 1 to about 40 carbon atoms and which may contain ether, thio or amine linkages in the main chain;
   * denotes a urethane or ureido linkage;
   a is at least 1;
   A independently denotes a divalent polymeric radical of Formula VI:

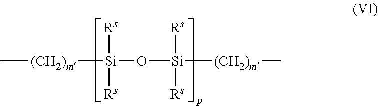

wherein each R^{s} independently denotes an alkyl or fluoro-substituted alkyl group having 1 to about 10 carbon atoms which may contain ether linkages between the carbon atoms; m′ is at least 1; and p is a number that provides a moiety weight of about 400 to about 10,000;
   each of E and E′ independently denotes a polymerizable unsaturated organic radical represented by Formula VII:

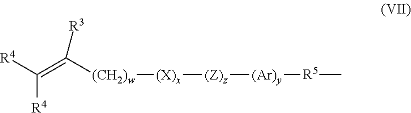

wherein: R³ is hydrogen or methyl;
R⁴ is hydrogen, an alkyl radical having 1 to 6 carbon atoms, or a —CO—Y—R⁶ radical wherein
Y is —O—, —S— or —NH—;
R⁵ is a divalent alkylene radical having 1 to about 10 carbon atoms;
R⁶ is a alkyl radical having 1 to about 12 carbon atoms;
X denotes —CO— or —OCO—;
Z denotes —O— or —NH—;
Ar denotes an aromatic radical having about 6 to about 30 carbon atoms;
w is 0 to 6; x is 0 or 1; y is 0 or 1; and z is 0 or 1.

The one or more silicone monomers can further include, as a class of representative silicone monomers, one or more silicone-containing urethane monomers represented by Formula VIII:

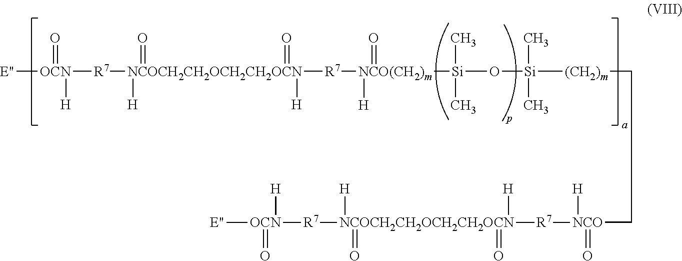

wherein m is at least 1 and is preferably 3 or 4, a is at least 1 and preferably is 1, p is a number which provides a moiety weight of about 400 to about 10,000 and is preferably at least about 30, R⁷ is a diradical of a diisocyanate after removal of the isocyanate group, such as the diradical of isophorone diisocyanate, and each E″ is a group represented by:

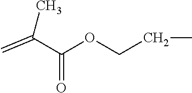

In another embodiment, a silicone hydrogel material comprises (in bulk, that is, in the monomeric mixture that is copolymerized) about 5 to about 50 percent, or from about 10 to about 25, by weight of one or more silicone macromonomers, about 5 to about 75 percent, or about 30 to about 60 percent, by weight of one or more polysiloxanylalkyl (meth)acrylic monomers, and about 10 to about 50 percent, or about 20 to about 40 percent, by weight of a hydrophilic monomer. In general, the silicone macromonomer is a poly(organosiloxane) capped with an unsaturated group at two or more ends of the molecule. In addition to the end groups in the above structural formulas, U.S. Pat. No. 4,153,641 discloses additional unsaturated groups, including acryloxy or methacryloxy. Fumarate-containing materials such as those disclosed in U.S. Pat. Nos. 5,310,779; 5,449,729 and 5,512,205 are also suitable substrates in accordance with the non-limiting embodiments described herein. The silane macromonomer may be a silicone-containing vinyl carbonate or vinyl carbamate or a polyurethane-polysiloxane having one or more hard-soft-hard blocks and end-capped with a hydrophilic monomer.

The one or more silicone monomers can further include, as a class of representative silicone monomers, one or more monomers of Formula IX:

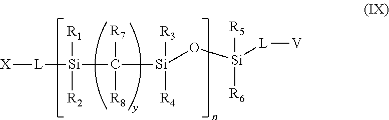

wherein X is the residue of a ring opening agent; L is the same or different and is a linking group or a bond; V is an ethylenically unsaturated polymerizable group; R₁, R₂, R₃, R₄, R₅, R₆ are independently hydrogen, an alkyl group, a haloalkyl group, a cycloalkyl group, a heterocycloalkyl group, an alkenyl group, a halo alkenyl group, or an aromatic group; R₇ and R₈ are independently hydrogen or an alkyl group wherein at least one of R₇ or R₈ is hydrogen; y is 2-7 and n is 1-100.

Ring opening agents are well known in the literature. Non-limiting examples of anionic ring opening agents include alkyl lithium, an alkoxide, trialkylsiloxylithium wherein the alkyl group may or may not contain halo atoms.

Linking groups can be any divalent radical or moiety and include substituted or unsubstituted alkyl, alkyl ether, alkenyls, alkenyl ethers, halo alkyls, substituted or unsubstituted siloxanes, and monomers capable of propagating ring opening.

Ethylenically unsaturated polymerizable groups are well known to those skilled in the art and can be any of those discussed above.

The one or more silicone monomers can further include, as a class of representative silicone monomers, one or more monomers of Formula X:

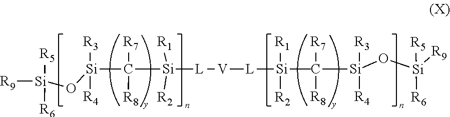

wherein L is the same or different and is a linking group or a bond; V is the same or different and is an ethylenically unsaturated polymerizable group; R₁, R₂, R₃, R₄, R₅, R₆ and R₉ are independently hydrogen, an alkyl group, a haloalkyl group, a cycloalkyl group, a heterocycloalkyl group, an alkenyl group, a halo alkenyl group, or an aromatic group; R₇ and R₈ are independently hydrogen or an alkyl group wherein at least one of R₇ or R₈ is hydrogen; y is 2-7 and n is 1-100.

The one or more silicone monomers can further include, as a class of representative silicone monomers, one or more monomers of Formulae XI and XII:

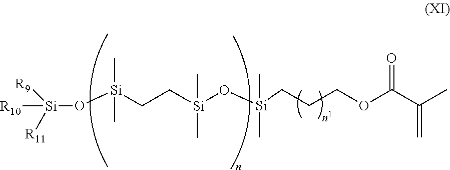

wherein R₉, R₁₀ and R₁₁ are independently hydrogen, an alkyl group, a haloalkyl group or other substituted alkyl groups; n is as defined above and n¹ is 0-10; and,

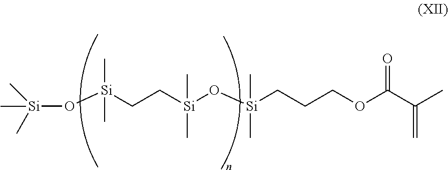

wherein n is 1 to 100, or n is 2 to 80, or n is 3 to 20, or n is 5 to 15.

The one or more silicone monomers can further include, as a class of representative silicone monomers, one or more monomers of Formulas XIII-XVII:

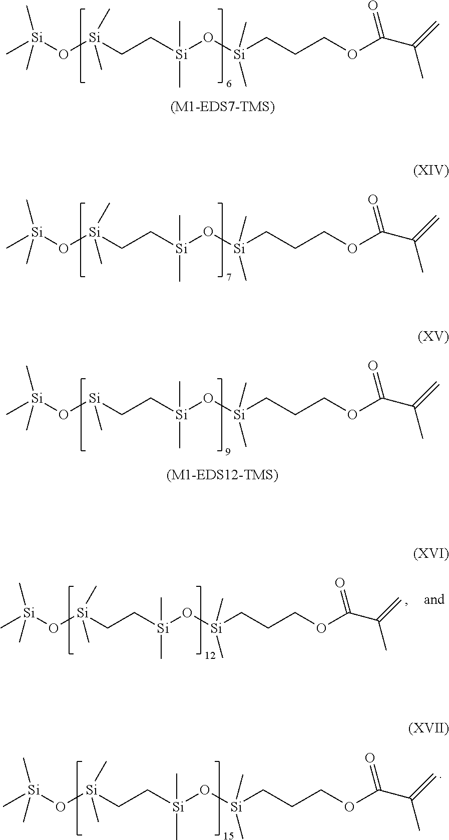

The one or more silicone monomers can further include, as a class of representative silicone monomers, one or more monomers of Formulas XVIII-XX:

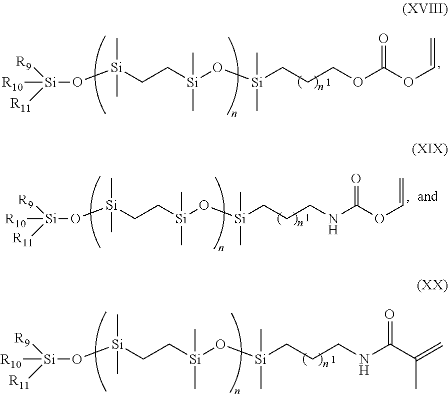

wherein R₉, R₁₀ and R₁₁ are independently hydrogen, an alkyl group, a haloalkyl group or other substituted alkyl groups and n and n¹ are as defined above.

The one or more silicone monomers can further include, as a class of representative silicone monomers, one or more monomers of Formulas XXI-XXIII:

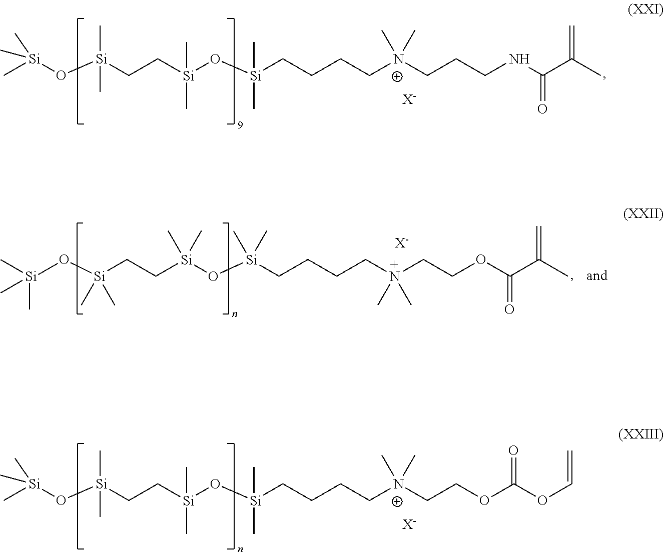

wherein n is as defined above and X⁻ is a counterion to provide an overall neutral charge.

Counterions capable of providing an overall neutral charge are well known to those of ordinary skill in the art and would include, for example, halide ions.

The one or more silicone monomers can further include, as a class of representative silicone monomers, one or more monomers of Formula XXIV:

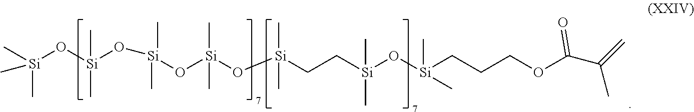

The above silicone materials are merely exemplary, and other materials for use as substrates that have been disclosed in various publications and are being continuously developed for use in contact lenses and other silicone hydrogels can also be used. For example, a silicone hydrogel can be formed from at least a cationic monomer such as cationic silicone-containing monomers or cationic fluorinated silicone-containing monomers.

The one or more silicone monomers can be present in the monomeric mixture in an amount ranging from about 30 wt. % to about 90 wt. %, based on the total weight of the monomeric mixture. In some embodiments, the one or more silicone monomers can be present in the monomeric mixture in an amount ranging from about 30 wt. % to about 70 wt. %, based on the total weight of the monomeric mixture.

The monomeric mixture can further contain one or more hydrophilic comonomers. Suitable hydrophilic comonomers include, for example, unsaturated carboxylic acids, acrylamides, vinyl lactams, hydroxyl-containing-(meth)acrylates, hydrophilic vinyl carbonates, hydrophilic vinyl carbamates, hydrophilic oxazolones, and poly(alkene glycols) functionalized with polymerizable groups and the like and mixtures thereof. Representative examples of unsaturated carboxylic acids include, but are not limited to, methacrylic acid, acrylic acid and the like and mixtures thereof. Representative examples of acrylamides include, but are not limited to, alkylamides such as N,N-dimethylacrylamide, N,N-dimethylmethacrylamide and the like and mixtures thereof. Representative examples of cyclic lactams include, but are not limited to, N-vinyl-2-pyrrolidone, N-vinyl caprolactam, N-vinyl-2-piperidone and the like and mixtures thereof. Representative examples of hydroxyl-containing (meth)acrylates include, but are not limited to, 2-hydroxyethyl methacrylate (HEMA), glycerol methacrylate and the like and mixtures thereof.

Additional hydrophilic comonomers include, for example, the hydrophilic vinyl carbonate or vinyl carbamate monomers disclosed in U.S. Pat. No. 5,070,215, and the hydrophilic oxazolone monomers disclosed in U.S. Pat. No. 4,910,277. Other suitable silicone hydrogel-forming hydrophilic comonomers will be apparent to one skilled in the art. Mixtures of the foregoing hydrophilic comonomers can also be used in the monomeric mixtures herein.

The one or more hydrophilic comonomers can be present in the monomeric mixture in an amount ranging from about 5 wt. % to about 95 wt. %, based on the total weight of the monomeric mixture. In some embodiments, the one or more hydrophilic comonomers can be present in the monomeric mixture in an amount ranging from about 5 wt. % to about 60 wt. %, based on the total weight of the monomeric mixture.

The monomeric mixture can further include one or more hydrophobic monomers. Suitable hydrophobic monomers include, for example, substitute or unsubstituted C₁ to C₂₀ alkyl and C₃ to C₂₀ cycloalkyl (meth)acrylates such as (2-amino)ethyl methacrylate and methacrylic acid, substituted and unsubstituted aryl (meth)acrylates (wherein the aryl group comprises 6 to 36 carbon atoms), (meth) acrylonitrile, styrene, lower alkyl styrene, lower alky vinyl ethers, and C₂ to C₁₀ perfluroalkyl (meth)acrylates and correspondingly partially fluorinate (meth)acrylates.

The one or more hydrophobic monomers can be present in the monomeric mixture in an amount ranging from about 5 wt. % to about 95 wt. %, based on the total weight of the monomeric mixture. In some embodiments, the one or more hydrophobic monomers can be present in the monomeric mixture in an amount ranging from about 10 wt. % to about 80 wt. %, based on the total weight of the monomeric mixture.

The monomeric mixture can further contain one or more crosslinking agents. The crosslinking agents for use herein are known in the art. Suitable one or more cross-linking agents include, for example, one or more crosslinking agents containing at least two ethylenically unsaturated reactive end groups. In some embodiments, the ethylenically unsaturated reactive end groups are (meth)acrylate-containing reactive end groups. In another embodiment, the ethylenically unsaturated reactive end groups are non-(meth)acrylate reactive end groups. In some embodiments, the ethylenically unsaturated reactive end groups are a combination of one or more (meth)acrylate-containing reactive end groups and one or more non-(meth)acrylate reactive end groups.

In an illustrative embodiment, suitable one or more crosslinking agents containing at least two ethylenically unsaturated reactive end groups include, for example, one or more di-, tri- or tetra(meth)acrylate-containing crosslinking agents. In an illustrative embodiment, suitable one or more di-, tri- or tetra(meth)acrylate-containing crosslinking agents include, for example, alkanepolyol di-, tri- or tetra(meth)acrylate-containing crosslinking agents such as, for example, one or more alkylene glycol di(meth)acrylate crosslinking agents, one or more alkylene glycol tri(meth)acrylate crosslinking agents, one or more alkylene glycol tetra(meth)acrylate crosslinking agents, one or more alkanediol di(meth)acrylate crosslinking agents, alkanediol tri(meth)acrylate crosslinking agents, alkanediol tetra(meth)acrylate crosslinking agents, agents, one or more alkanetriol di(meth)acrylate crosslinking agents, alkanetriol tri(meth)acrylate crosslinking agents, alkanetriol tetra(meth)acrylate crosslinking agents, agents, one or more alkanetetraol di(meth)acrylate crosslinking agents, alkanetetraol tri(meth)acrylate crosslinking agents, alkanetetraol tetra(meth)acrylate crosslinking agents and the like and mixtures thereof.

In an illustrative embodiment, one or more alkylene glycol di(meth)acrylate crosslinking agents include tetraethylene glycol dimethacrylate, ethylene glycol di(meth)acrylates having up to about 10 ethylene glycol repeating units, butyleneglycol di(meth)acrylate and the like. In some embodiments, one or more alkanediol di(meth)acrylate crosslinking agents include butanediol di(meth)acrylate crosslinking agents, hexanediol di(meth)acrylate and the like. In some embodiments, one or more alkanetriol tri(meth)acrylate crosslinking agents are trimethylol propane trimethacrylate crosslinking agents. In some embodiments, one or more alkanetetraol tetra(meth)acrylate crosslinking agents are pentaerythritol tetramethacrylate crosslinking agents.

In a non-limiting illustrative embodiment, suitable crosslinking agents include, for example, ethylene glycol diacrylate, diethylene glycol diacrylate, allyl acrylate, 1,3-propanediol diacrylate, 2,3-propanediol diacrylate, 1,6-hexanediol diacrylate, 1,4-butanediol diacrylate, triethylene glycol diacrylate, cyclohexane-1,1-diyldimethanol diacrylate, 1,4-cyclohexanediol diacrylate, 1,3-adamantanediol diacrylate, 1,3-adamantanedimethyl diacrylate, 2,2-diethyl-1,3-propanediol diacrylate, 2,2-diisobutyl-1,3-propanediol diacrylate, 1,3-cyclohexanedimethyl diacrylate, 1,4-cyclohexanedimethyl diacrylate; neopentyl glycol diacrylate, tetraethyleneglycol diacrylate, polyethyleneglycol diacrylate; and their corresponding methacrylates.

In a non-limiting illustrative embodiment, suitable crosslinking agents include, for example, 1,4-butanediol diacrylate, 1,4-butanediol dimethacrylate, ethylene glycol dimethacrylate, diethylene glycol diacrylate, diethylene glycol dimethacrylate, triethylene glycol diacrylate, triethylene glycol dimethacrylate, tetraethylene glycol diacrylate, tetraethylene glycol dimethacrylate, 1,3-propanediol diacrylate, 1,3-propanediol dimethacrylate, 1,6-hexanediol diacrylate, 1,6-hexanediol dimethacrylate, poly(ethylene glycol) diacrylate (Mn=700 Daltons), poly(ethylene glycol) dimethacrylate (Mn=700 Daltons), and poly(ethylene glycol) dimethacrylate (Mn=1000 Daltons).

In some embodiments, the one or more crosslinking agents containing at least two ethylenically unsaturated reactive end groups include at least one allyl-containing reactive end group and at least one (meth)acrylate-containing reactive end group. For example, the one or more crosslinking agents can be allyl methacrylate.

The one or more crosslinking agents are present in the monomeric mixture in an amount of about 0.1 wt. % to about 3.0 wt. %, based on the total weight of the monomeric mixture. In some embodiments, the one or more crosslinking agents are present in the monomeric mixture in an amount of about 0.2 wt. % to about 1.0 wt. %, based on the total weight of the monomeric mixture.

In non-limiting illustrative embodiments, as may be combined with one or more of the preceding paragraphs, the monomeric mixture can further contain a reactive (polymerizable) ultraviolet (UV) light absorber and/or a reactive blue-light absorber. Suitable reactive UV light absorbers can be any known reactive UV light absorber. In non-limiting illustrative embodiments, suitable reactive UV light absorbers include, for example, 2-(2′-hydroxy-3′-methallyl-5′-methylphenyl)benzotriazole, commercially available as o-Methallyl Tinuvin P (“oMTP”) from Polysciences, Inc., Warrington, Pa., 3-(2H-benzo[d][1,2,3]triazol-2-yl)-4-hydroxyphenylethyl methacrylate, and 2-(3-(tert-butyl)-4-hydroxy-5-(5-methoxy-2H-benzo[d][1,2,3]triazol-2-yl)phenoxy)ethyl methacrylate.

In one illustrative embodiment, suitable UV light absorbers include, for example, one or more compounds of the following Formulas:

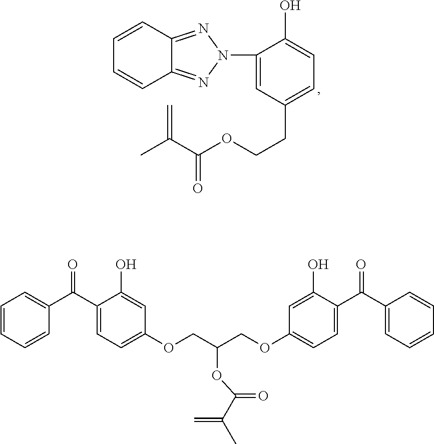 [SMILES: C=C(C)C(=O)OCCc1ccc(O)c(-n2nc3ccccc3n2)c1 ; C=C(C)C(=O)OC(COc1ccc(C(=O)c2ccccc2)c(O)c1)COc1ccc(C(=O)c2ccccc2)c(O)c1]

(2-Propenoic acid, 2-methyl,2-(4-benzoyl-3-hydroxyphenoxy)-1-[(4-benzoyl3-hydroxyphenoxy)methyl ester),

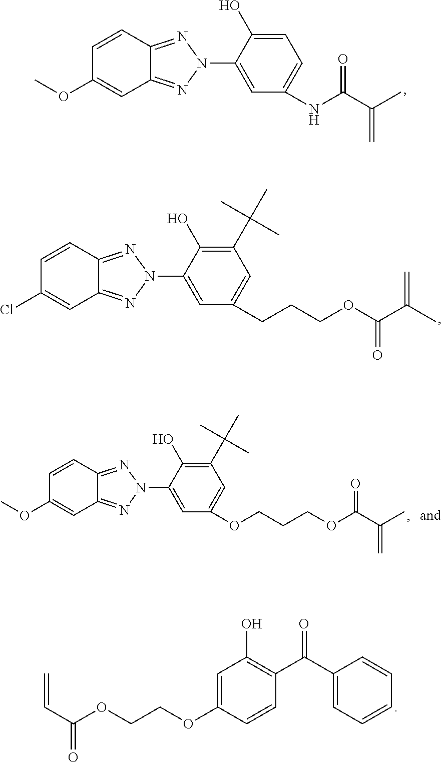

These compounds are merely illustrative and not intended to be limiting. Any known UV blocker or later developed UV blocker is contemplated for use herein.

The UV light absorbers can be present in the monomeric mixture in an amount ranging from about 0.1 wt. % to about 5 wt. %, based on the total weight of the monomeric mixture. In some embodiments, the UV light absorbers can be present in the monomeric mixture in an amount ranging from about 1.5 wt. % to about 2.5 wt. %, based on the total weight of the monomeric mixture. In some embodiments, the UV light absorbers can be present in the monomeric mixture in an amount ranging from about 1.5 wt. % to about 2 wt. %, based on the total weight of the monomeric mixture.

Many reactive blue-light absorbing compounds are known. Preferred reactive blue-light absorbing compounds are those described in U.S. Pat. Nos. 5,470,932; 8,207,244; and 8,329,775, the contents of which are hereby incorporated by reference. In some embodiments, a blue-light absorbing dye is N-2-[3-(2′-methylphenylazo)-4-hydroxyphenyl]ethyl methacrylamide. The blue-light absorbers can be present in the monomeric mixture in an amount ranging from about 0.005 wt. % to about 1 wt. %, based on the total weight of the monomeric mixture. In some embodiments, the blue-light absorbers can be present in the monomeric mixture in an amount ranging from about 0.01 wt. % to about 1 wt. %, based on the total weight of the monomeric mixture.

The monomeric mixture can further contain a diluent. Suitable diluents include, for example, at least one or more boric acid esters of a C₁ to C₈ monohydric alcohol, water-soluble or partly water-soluble monohydric alcohols and mixtures thereof. In some embodiments, a diluent includes, for example, at least one or more boric acid esters of a C₁ to C₅ monohydric alcohol. Suitable boric acid esters of a C₁ to C₈ monohydric alcohol include, for example, trimethyl borate, triethyl borate, tri-n-propyl borate, triisopropyl borate, tri-n-butyl borate, and tri-tert-butyl borate. Suitable water-soluble or partly water-soluble monohydric alcohols include, for example, monohydric alcohols having from 1 to 5 carbon atoms such as methanol, ethanol, isopropyl alcohol, 1-propanol, t-butyl alcohol, 2-butyl alcohol, 2-methyl-1-propanol, t-amyl alcohol and other C₅ isomers.

In non-limiting illustrative embodiments, as may be combined with one or more of the preceding paragraphs, the monomeric mixture contains about 5 wt. % to about 50 wt. % of the diluent, based on the total weight of the monomeric mixture. In some embodiments, the monomeric mixture contains about 15 wt. % to about 30 wt. % of the diluent, based on the total weight of the monomeric mixture.

The monomeric mixture may further contain, as necessary and within limits not to impair the purpose and effect of the illustrative embodiments, various additives such as an antioxidant, coloring agent, lubricant, internal wetting agent, toughening agent and the like and other constituents as are well known in the art.

The silicone ophthalmic devices of the illustrative embodiments, e.g., silicone contact lenses or intraocular lenses, can be prepared by polymerizing the foregoing monomeric mixtures to form a product that can be subsequently formed into the appropriate shape by, for example, lathing, injection molding, compression molding, cutting and the like. For example, in producing contact lenses, the initial mixture may be polymerized in tubes to provide rod-shaped articles, which are then cut into buttons. The buttons may then be lathed into contact lenses.

Alternately, the silicone ophthalmic devices such as silicone contact lenses may be cast directly in molds, e.g., polypropylene molds, from the mixtures, e.g., by spincasting and static casting methods. Spincasting methods are disclosed in U.S. Pat. Nos. 3,408,429 and 3,660,545, and static casting methods are disclosed in U.S. Pat. Nos. 4,113,224, 4,197,266, and 5,271,875. Spincasting methods involve charging the mixtures to be polymerized to a mold, and spinning the mold in a controlled manner while exposing the mixture to a radiation source such as UV light. Static casting methods involve charging the monomeric mixture between two mold sections, one mold section shaped to form the anterior lens surface and the other mold section shaped to form the posterior lens surface, and curing the mixture while retained in the mold assembly to form a lens, for example, by free radical polymerization of the mixture. Examples of free radical reaction techniques to cure the lens material include thermal radiation, infrared radiation, electron beam radiation, gamma radiation, ultraviolet (UV) radiation, and the like; or combinations of such techniques may be used. U.S. Pat. No. 5,271,875 describes a static cast molding method that permits molding of a finished lens in a mold cavity defined by a posterior mold and an anterior mold. As an additional method, U.S. Pat. No. 4,555,732 discloses a process where an excess of a monomeric mixture is cured by spincasting in a mold to form a shaped article having an anterior lens surface and a relatively large thickness, and the posterior surface of the cured spincast article is subsequently lathed to provide a contact lens having the desired thickness and posterior lens surface.

Polymerization may be facilitated by exposing the mixture to heat (thermal cure) and/or radiation, such as ultraviolet light, visible light, or high energy radiation. A polymerization initiator may be included in the mixture to facilitate the polymerization step. Representative examples of free radical thermal polymerization initiators include organic peroxides such as acetyl peroxide, lauroyl peroxide, decanoyl peroxide, stearoyl peroxide, benzoyl peroxide, tertiarylbutyl peroxypivalate, peroxydicarbonate, and the like. Representative examples of diazo initiators include VAZO 64, and VAZO 67. Representative UV initiators are those known in the art and include benzoin methyl ether, benzoin ethyl ether, Darocur® 1173, 1164, 2273, 1116, 2959, 3331 (EM Industries) and Irgacure® 651 and 184 (Ciba-Geigy). Representative visible light initiators include IRGACURE 819 and other phosphine oxide-type initiators, and the like. Generally, the initiator will be employed in the monomeric mixture at a concentration of about 0.01 wt. % to about 5 wt. % of the total mixture.

Generally, polymerization can be carried out for about 15 minutes to about 72 hours, and under an inert atmosphere of, for example, nitrogen or argon. If desired, the resulting polymerization product can be dried under vacuum, e.g., for about 5 to about 72 hours, or left in an aqueous solution prior to use.

Polymerization of the mixtures will yield a polymer, that when hydrated, forms a silicone hydrogel. When producing a silicone hydrogel lens, the mixture may further include at least a diluent as discussed above that is ultimately replaced with water when the polymerization product is hydrated to form a hydrogel. In non-limiting illustrative embodiments, as may be combined with one or more of the preceding paragraphs, the silicone hydrogels disclosed herein can be high water content silicone hydrogels having an equilibrium water content of at least about 50 wt. %. In another illustrative embodiment, the silicone hydrogels disclosed herein can be high water content silicone hydrogels having an equilibrium water content of at least about 60 wt. %. In another illustrative embodiment, the silicone hydrogels disclosed herein can be high water content silicone hydrogels having an equilibrium water content of at least about 70 wt. %. In another illustrative embodiment, the silicone hydrogels disclosed herein can be high water content silicone hydrogels having an equilibrium water content of from about 50 wt. % to about 80 wt. %.

### Hydrophilic Surface Coating

In non-limiting illustrative embodiments, as may be combined with one or more of the preceding paragraphs, the foregoing silicone ophthalmic devices are surface modified with a hydrophilic surface coating comprising a copolymer comprising (i) monomeric units derived from an ethylenically unsaturated-containing monomer having ring-opening reactive functionalities, and (ii) monomeric units derived from a hydrophilic monomer having an ethylenically unsaturated reactive group.

In some embodiments, the copolymer is a random copolymer. In a non-limiting illustrative embodiment, the random copolymer is a brush copolymer. The term “polymer brushes,” as used herein is understood to mean a polymer brush that contains polymer chains, one end of which is directly or indirectly tethered to a surface and another end of which is free to extend from the surface, somewhat analogous to the bristles of a brush. In some embodiments, the random copolymer is a linear copolymer.

In some embodiments, the copolymer is a block copolymer. In an illustrative embodiment, the block copolymer is a brush copolymer. In an illustrative embodiment, the block copolymer is a linear copolymer.

Representative examples of the ethylenically unsaturated moiety of the ethylenically unsaturated-containing monomer having ring-opening reactive functionalities include, by way of example, (meth)acrylate-containing radicals, (meth)acrylamido-containing radicals, vinylcarbonate-containing radicals, vinylcarbamate-containing radicals, styrene-containing radicals, itaconate-containing radicals, vinyl-containing radicals, vinyloxy-containing radicals, fumarate-containing radicals, maleimide-containing radicals, vinylsulfonyl radicals and the like.

In an illustrative embodiment, ethylenically unsaturated-containing monomers having ring-opening reactive functionalities that are complementary to the ophthalmic device surface reactive functional groups include ethylenically unsaturated epoxy-containing monomers. Suitable ethylenically unsaturated epoxy-containing monomers include, for example, glycidyl-containing ethylenically unsaturated monomers such as glycidyl methacrylate, glycidyl acrylate, glycidyl vinylcarbonate, glycidyl vinylcarbamate, vinylcyclohexyl-1,2-epoxide and the like. In some embodiments, an ethylenically unsaturated-containing monomer having ring-opening reactive functionalities can contain from 2 to about 18 carbon atoms which are substituted by an epoxy group. In some embodiments, the ethylenically unsaturated-containing monomer having ring-opening reactive functionalities that are complementary to the ophthalmic device surface reactive functional groups is glycidyl methacrylate.

The copolymers further include monomeric units derived from a hydrophilic monomer having an ethylenically unsaturated reactive group. Suitable ethylenically unsaturated reactive groups can be any of those discussed above. Suitable hydrophilic monomers having an ethylenically unsaturated reactive group include, for example, unsaturated carboxylic acids, acrylamides, vinyl lactams, hydroxyl-containing-(meth)acrylates, hydrophilic vinyl carbonates, hydrophilic vinyl carbamates, hydrophilic oxazolones, and poly(alkene glycols) functionalized with polymerizable groups and the like and mixtures thereof. Representative examples of unsaturated carboxylic acids include methacrylic acid, acrylic acid and the like and mixtures thereof. Representative examples of amides include alkylamides such as N,N-dimethylacrylamide, N,N-dimethylmethacrylamide and the like and mixtures thereof. Representative examples of cyclic lactams include N-vinyl-2-pyrrolidone, N-vinyl caprolactam, N-vinyl-2-piperidone and the like and mixtures thereof. Representative examples of hydroxyl-containing (meth)acrylates include 2-hydroxyethyl methacrylate (HEMA), glycerol methacrylate and the like and mixtures thereof. Still further examples are the hydrophilic vinyl carbonate or vinyl carbamate monomers disclosed in U.S. Pat. No. 5,070,215, and the hydrophilic oxazolone monomers disclosed in U.S. Pat. No. 4,910,277. Other suitable hydrophilic monomers will be apparent to one skilled in the art. Mixtures of the foregoing hydrophilic monomers can also be used in the monomeric mixtures herein.

In some embodiments, a hydrophilic monomer having an ethylenically unsaturated reactive group can be vinyl chloroformate. The vinyl chloroformate has vinyl groups for attaching to the backbone of the copolymer and a chloro group can be subsequently reacted with an OH group or an amine group to provide a functional derivative, e.g., vinyl chloroformate (Cl—COOCH═CH2) that can provide a —NH—COOCH═CH₂ functional derivative or a —O—COOCH═CH₂ functional derivative depending on the reaction with the NH₂— or OH— group, respectively.

In some embodiments, a hydrophilic monomer having an ethylenically unsaturated reactive group is an alkylacrylamide monomer. Suitable alkylacrylamides include, for example, N,N-dimethylacrylamide, N,N-dimethylmethacrylamide, and the like.

A representative example of a copolymer disclosed herein can be represented by the following structure:

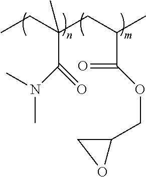

where n is from about 75 to about 95 and m is from about 5 to about 25.

The copolymers can be prepared using free radical polymerization techniques with the structure of the polymer being completely random or controlled by the reactivity ratios of the respective monomers.

In some embodiments, a random copolymer can be obtained by (1) mixing the ethylenically unsaturated-containing monomer having ring-opening reactive functionalities and the hydrophilic monomer, (2) adding a polymerization initiator, (3) and subjecting the monomer/initiator mixture to a source of heat. The amount of the ethylenically unsaturated-containing monomer having ring-opening reactive functionalities in the mixture can range from about 10 mol % to about 25 mol % and the amount of the hydrophilic monomer can range from about 75 mol % to about 90 mol %.

Suitable initiators can be any known initiator such as, for example, free-radical-generating polymerization initiators of the type illustrated by acetyl peroxide, lauroyl peroxide, decanoyl peroxide, caprylyl peroxide, benzoyl peroxide, tertiary butyl peroxypivalate, sodium percarbonate, tertiary butyl peroctoate, and azobis-isobutyronitrile (AIBN). A level of initiator employed will vary within the range of 0.01 wt. % to 2 wt. %, based on the total weight of the mixture. If desired, the mixture of the above-mentioned monomers is warmed with addition of a free-radical former.

In some embodiments, the reaction can be carried out until all of the ethylenically unsaturated-containing monomer having ring-opening reactive functionalities has been reacted. Suitable polymerization conditions include, for example, a temperature of between about 15° C. to about 120° C. for a time period of about 30 minutes to about 48 hours. If desired, the reaction can be carried out in the presence of a suitable solvent. Suitable solvents are in principle all solvents which dissolve the monomers used including, for example, 1,4-dioxane, hexanol, dimethylformamide; acetone, cyclohexanone, toluene, and the like and mixtures thereof.

The copolymers disclosed herein can also be prepared using techniques of controlled radical polymerization, e.g., by reversible addition-fragmentation chain transfer (RAFT) polymerization or atom-transfer radical polymerization (ATRP) employing a chain transfer agent that allows construction of copolymers with a well-defined molecular weight distribution and narrow polydispersity. RAFT polymerization is particularly preferred because it is compatible with a wide variety of vinyl monomers.

In non-limiting illustrative embodiments, the RAFT agents suitable for use herein can be based upon thio carbonyl thio chemistry which is well known to those of ordinary skill in the art. The thio carbonyl thio fragment can be derived from a RAFT agent such as, for example, a xanthate-containing compound, trithiocarbonate-containing compound, dithiocarbamate-containing compound, a dithiobenzoate-containing compound or dithio ester-containing compound, wherein each compound contains a thio carbonyl thio group. One class of RAFT agents that can be used herein is of the general formula:

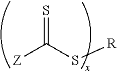

wherein x is 1 or 2, Z is a substituted oxygen (e.g., xanthates (—O—R)), a substituted nitrogen (e.g., dithiocarbamates (—NRR)), a substituted sulfur (e.g., trithiocarbonates (—S—R)), a dithiobenzoate, a substituted or unsubstituted C₁-C₂₀ alkyl or C₃-C₂₅ unsaturated, or partially or fully saturated ring (e.g., dithioesters (—R)) or carboxylic acid-containing group; and R is independently a straight or branched, substituted or unsubstituted C₁-C₃₀ alkyl cyano group, a straight or branched, substituted or unsubstituted C₁-C₃₀ alkyl group, a substituted or unsubstituted C₃-C₃₀ cycloalkyl group, a substituted or unsubstituted C₃-C₃₀ cycloalkylalkyl group, a substituted or unsubstituted C₃-C₃₀ cycloalkenyl group, a substituted or unsubstituted C₅-C₃₀ aryl group, a substituted or unsubstituted C₅-C₃₀ arylalkyl group, a C₁-C₂₀ ester group; an ether or polyether-containing group; an alkyl- or arylamide group; an alkyl- or arylamine group; a substituted or unsubstituted C₅-C₃₀ heteroaryl group; a substituted or unsubstituted C₃-C₃₀ heterocyclic ring; a substituted or unsubstituted C₄-C₃₀ heterocycloalkyl group; a substituted or unsubstituted C₆-C₃₀ heteroarylalkyl group; and combinations thereof.

Representative examples of alkyl groups for use herein include, by way of example, a straight or branched alkyl chain radical containing carbon and hydrogen atoms of from 1 to about 30 carbon atoms and preferably from 1 to about 12 carbon atoms with or without unsaturation, to the rest of the molecule, e.g., methyl, ethyl, n-propyl, 1-methylethyl (isopropyl), n-butyl, n-pentyl, methylene, ethylene, etc., and the like.

Representative examples of cycloalkyl groups for use herein include, by way of example, a substituted or unsubstituted non-aromatic mono or multicyclic ring system of about 3 to about 30 carbon atoms and preferably from 3 to about 6 carbon atoms such as, for example, cyclopropyl, cyclobutyl, cyclopentyl, cyclohexyl, perhydronapththyl, adamantyl and norbornyl groups, bridged cyclic groups or sprirobicyclic groups, e.g., spiro-(4, 4)-non-2-yl and the like, optionally containing one or more heteroatoms, e.g., O and N, and the like.

Representative examples of cycloalkylalkyl groups for use herein include, by way of example, a substituted or unsubstituted cyclic ring-containing radical containing from about 3 to about 30 carbon atoms and preferably from 3 to about 6 carbon atoms directly attached to the alkyl group which are then attached to the main structure of the monomer at any carbon from the alkyl group that results in the creation of a stable structure such as, for example, cyclopropylmethyl, cyclobutylethyl, cyclopentylethyl and the like, wherein the cyclic ring can optionally contain one or more heteroatoms, e.g., O and N, and the like.

Representative examples of cycloalkenyl groups for use herein include, by way of example, a substituted or unsubstituted cyclic ring-containing radical containing from about 3 to about 30 carbon atoms and preferably from 3 to about 6 carbon atoms with at least one carbon-carbon double bond such as, for example, cyclopropenyl, cyclobutenyl, cyclopentenyl and the like, wherein the cyclic ring can optionally contain one or more heteroatoms, e.g., O and N, and the like.

Representative examples of aryl groups for use herein include, by way of example, a substituted or unsubstituted monoaromatic or polyaromatic radical containing from about 5 to about 30 carbon atoms such as, for example, phenyl, naphthyl, tetrahydronapthyl, indenyl, biphenyl and the like, optionally containing one or more heteroatoms, e.g., O and N, and the like.

Representative examples of arylalkyl groups for use herein include, by way of example, a substituted or unsubstituted aryl group as defined herein directly bonded to an alkyl group as defined herein, e.g., —CH₂C₆H₅, —C₂H₅C₆H₅ and the like, wherein the aryl group can optionally contain one or more heteroatoms, e.g., O and N, and the like.

Representative examples of ester groups for use herein include, by way of example, a carboxylic acid ester having one to 20 carbon atoms and the like.

Representative examples of ether or polyether containing groups for use herein include, by way of example, an alkyl ether, cycloalkyl ether, cycloalkylalkyl ether, cycloalkenyl ether, aryl ether, arylalkyl ether wherein the alkyl, cycloalkyl, cycloalkylalkyl, cycloalkenyl, aryl, and arylalkyl groups are as defined herein. Exemplary ether or polyether-containing groups include, by way of example, alkylene oxides, poly(alkylene oxide)s such as ethylene oxide, propylene oxide, butylene oxide, poly(ethylene oxide)s, poly(ethylene glycol)s, poly(propylene oxide)s, poly(butylene oxide)s and mixtures or copolymers thereof, an ether or polyether group of the general formula —(R²OR³)ₜ, wherein R² is a bond, a substituted or unsubstituted alkyl, cycloalkyl or aryl group as defined herein and R³ is a substituted or unsubstituted alkyl, cycloalkyl or aryl group as defined herein and t is at least 1, e.g., —CH₂CH₂OC₆H₅ and CH₂—CH₂—CH₂—O—CH₂—(CF₂)_{z}—H where z is 1 to 6, —CH₂CH₂OC₂H₅, and the like.

Representative examples of alkyl or arylamide groups for use herein include, by way of example, an amide of the general formula —R⁴C(O)NR⁵R⁶ wherein R⁴, R⁵ and R⁶ are independently C₁-C₃₀ hydrocarbons, e.g., R⁴ can be alkylene groups, arylene groups, cycloalkylene groups and R⁵ and R⁶ can be alkyl groups, aryl groups, and cycloalkyl groups as defined herein and the like.

Representative examples of alky or arylamine groups for use herein include, by way of example, an amine of the general formula —R⁷NR⁸R⁹ wherein R⁷ is a C₂-C₃₀ alkylene, arylene, or cycloalkylene and R⁸ and R⁹ are independently C₁-C₃₀ hydrocarbons such as, for example, alkyl groups, aryl groups, or cycloalkyl groups as defined herein.

Representative examples of heterocyclic ring groups for use herein include, by way of example, a substituted or unsubstituted stable 3 to about 30 membered ring radical, containing carbon atoms and from one to five heteroatoms, e.g., nitrogen, phosphorus, oxygen, sulfur and mixtures thereof. Suitable heterocyclic ring radicals for use herein may be a monocyclic, bicyclic or tricyclic ring system, which may include fused, bridged or spiro ring systems, and the nitrogen, phosphorus, carbon, oxygen or sulfur atoms in the heterocyclic ring radical may be optionally oxidized to various oxidation states. In addition, the nitrogen atom may be optionally quaternized; and the ring radical may be partially or fully saturated (i.e., heteroaromatic or heteroaryl aromatic).

Representative examples of heteroaryl groups for use herein include, by way of example, a substituted or unsubstituted heterocyclic ring radical as defined herein. The heteroaryl ring radical may be attached to the main structure at any heteroatom or carbon atom that results in the creation of a stable structure.

Representative examples of heteroarylalkyl groups for use herein include, by way of example, a substituted or unsubstituted heteroaryl ring radical as defined herein directly bonded to an alkyl group as defined herein. The heteroarylalkyl radical may be attached to the main structure at any carbon atom from the alkyl group that results in the creation of a stable structure.

Representative examples of heterocyclic groups for use herein include, by way of example, a substituted or unsubstituted heterocylic ring radical as defined herein. The heterocyclic ring radical may be attached to the main structure at any heteroatom or carbon atom that results in the creation of a stable structure.

Representative examples of heterocycloalkyl groups for use herein include, by way of example, a substituted or unsubstituted heterocylic ring radical as defined herein directly bonded to an alkyl group as defined herein. The heterocycloalkyl radical may be attached to the main structure at any carbon atom in the alkyl group that results in the creation of a stable structure.

The substituents in the ‘substituted oxygen’, ‘substituted nitrogen’, ‘substituted sulfur’, ‘substituted alkyl’, ‘substituted alkylene, ‘substituted cycloalkyl’, ‘substituted cycloalkylalkyl’, ‘substituted cycloalkenyl’, ‘substituted arylalkyl’, ‘substituted aryl’, ‘substituted heterocyclic ring’, ‘substituted heteroaryl ring,’ ‘substituted heteroarylalkyl’, ‘substituted heterocycloalkyl ring’, ‘substituted cyclic ring’ may be the same or different and include one or more substituents such as hydrogen, hydroxy, halogen, carboxyl, cyano, nitro, oxo (═O), thio(═S), substituted or unsubstituted alkyl, substituted or unsubstituted alkoxy, substituted or unsubstituted alkenyl, substituted or unsubstituted alkynyl, substituted or unsubstituted aryl, substituted or unsubstituted arylalkyl, substituted or unsubstituted cycloalkyl, substituted or unsubstituted cycloalkenyl, substituted or unsubstituted amino, substituted or unsubstituted aryl, substituted or unsubstituted heteroaryl, substituted heterocycloalkyl ring, substituted or unsubstituted heteroarylalkyl, substituted or unsubstituted heterocyclic ring, and the like.

Representative examples of RAFT agents for use herein include, but are not limited to, 4-cyano-4-(dodecyl-sulfanylthiocarbonyl)sulfanylpentanoic acid, S-cyanomethyl-5-dodecyltrithiocarbonate, S-(2-cyano-2-propyl)-S-dodecyltrithiocarbonate, 3-benzylsulfanylthiocarbonylsulfanyl-propionic acid, cumyl dithiobenzoate, 2-cyanoprop-2-yl dithiobenzoate (i.e., cyanoisopropyl dithiobenzoate), 4-thiobenzoylsulfanyl-4-cyanopentanoic acid (TCA), S,S′-bis(α,α′-dimethyl-alpha″-acetic acid)-trithiocarbonate (BATC), benzyl dodecyl trithiocarbonate, ethyl-2-dodecyl trithiocarbony) proprionate, S-sec propionic acid O-ethyl xanthate, α-ethyl xanthylphenylacetic acid, ethyl α-(o-ethyl xanthyl) proprionate, ethyl α-(ethyl xanthyl) phenyl acetate, ethyl 2-(dodecyl trithiocarbonyl) phenyl acetate, ethyl 2-(dodecyl trithiocarbonyl) propionate, 2-(dodecylthiocarbonylthiol)propanoic acid, and the like and mixtures thereof.

There is no particular limitation on the organic chemistry used to form the RAFT agent and it is within the purview of one skilled in the art.

The copolymers disclosed herein can be obtained in a first step (a) by (1) mixing either the ethylenically unsaturated-containing monomer having ring-opening reactive functionalities or the hydrophilic monomer with a RAFT agent; (2) adding a polymerization initiator; (3) and subjecting the monomer/RAFT agent/initiator mixture to a source of heat. Suitable initiators include, for example, free-radical-generating polymerization initiators of the type illustrated by acetyl peroxide, lauroyl peroxide, decanoyl peroxide, coprylyl peroxide, benzoyl peroxide, tertiary butyl peroxypivalate, sodium percarbonate, tertiary butyl peroctoate, and azobisisobutyronitrile (AIBN).

The reaction can be carried out at a temperature of between about 15° C. to about 120° C. for a time period of about 30 minutes to about 48 hours. If desired, the reaction can be carried out in the presence of a suitable solvent. Suitable solvents are in principle all solvents which dissolve the monomer used, for example, 1,4-dioxane, hexanol, dimethylformamide; acetone, cyclohexanone, toluene, and the like and mixtures thereof.

In an illustrative embodiment, the ethylenically unsaturated-containing monomer having ring-opening reactive functionalities or the hydrophilic monomer is employed in an amount ranging from about 10 wt. % to about 50 wt. %, based on the total weight of the mixture. In some embodiments, the RAFT agent is employed in an amount ranging from about 0.5 wt. % to about 3 wt. %, based on the total weight of the mixture. The level of initiator employed will vary within the range of 0.01 wt. % to 2 wt. % of the mixture of monomers. If desired, the mixture of the above-mentioned monomers is warmed with addition of a free-radical former.

Next, in step (b) the resulting product of step (a) is then mixed with the other one of the ethylenically unsaturated-containing monomers having ring-opening reactive functionalities or the hydrophilic monomer and an initiator and subjected to a source of heat as described above until the desired copolymer is formed. In an illustrative embodiment, the other one of the ethylenically unsaturated-containing monomers having ring-opening reactive functionalities or the hydrophilic monomer is employed in an amount ranging from about 10 wt. % to about 50 wt. %, based on the total weight of the mixture. In an illustrative embodiment, the resulting product of step (a) is employed in an amount ranging from about 1 wt. % to about 20 wt. %, based on the total weight of the mixture.

A non-limiting schematic representation of a synthetic method for making the block copolymer with a RAFT agent is set forth below in Scheme II:

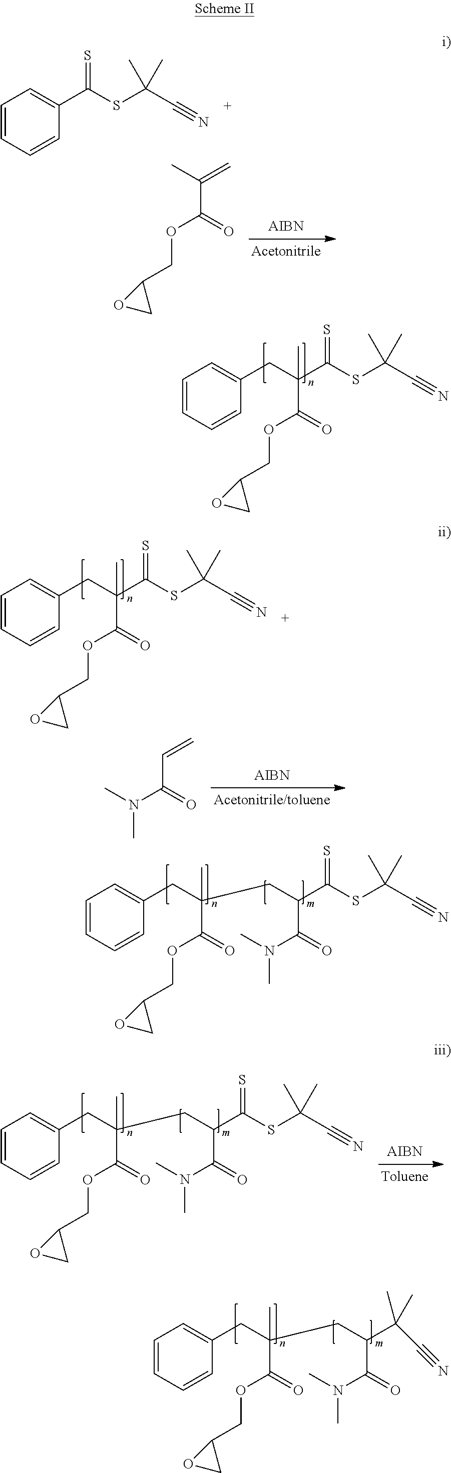

where m is about 75 to about 95, and n is about 5 to about 25.

As one skilled in the art will readily appreciate, the copolymer disclosed herein can contain a balance of monomeric units derived from the ethylenically unsaturated-containing monomer having ring-opening reactive functionalities and monomeric units derived from the hydrophilic monomer having an ethylenically unsaturated reactive group. In some embodiments, a copolymer can include from about 5 mol % to about 25 mol % of repeating units of the monomeric units derived from an ethylenically unsaturated-containing monomer having ring-opening reactive functionalities and from about 75 mol % to about 95 mol % of repeating units of the monomeric units derived from a hydrophilic monomer. In some embodiments, a copolymer can include from about 10 mol % to about 20 mol % of repeating units of the monomeric units derived from an ethylenically unsaturated-containing monomer having ring-opening reactive functionalities and from about 80 mol % to about 90 mol % of repeating units of the monomeric units derived from a hydrophilic monomer.

Any combination of the foregoing ranges of numbers of monomeric units derived from ethylenically unsaturated-containing monomers having ring-opening reactive functionalities and numbers of monomeric units derived from a hydrophilic monomer are contemplated herein.

### Surface-Modified Ophthalmic Device

In one or more additional non-limiting illustrative embodiments, as may be combined with one or more of the preceding paragraphs, a silicon ophthalmic device according to the present disclosure is surface modified by attaching the hydrophilic surface coating to a surface of the silicone ophthalmic device by van der Waals dispersion forces. In some embodiments, a silicon ophthalmic device is surface modified by attaching the hydrophilic surface coating to an anterior surface and to a posterior surface of the silicone ophthalmic device by van der Waals dispersion forces. In some embodiments, the hydrophilic surface coating has a uniform thickness on an anterior surface of the silicone ophthalmic device and on the posterior surface of the silicone ophthalmic device. In some embodiments, the hydrophilic surface coating has a thickness of about 0.1 micrometers (μm) to about 2 μm on an anterior surface of the silicone ophthalmic device and a thickness of about 0.1 μm to about 2 μm on the posterior surface of the silicone ophthalmic device.

For example, in non-limiting illustrative embodiments, attaching the hydrophilic surface coating to a surface of the silicone ophthalmic device can comprise immersing a silicon ophthalmic device in an aqueous solution comprising a copolymer comprising (i) monomeric units derived from an ethylenically unsaturated-containing monomer having ring-opening reactive functionalities, and (ii) monomeric units derived from a hydrophilic monomer having an ethylenically unsaturated reactive group and heating to temperature and for a time period sufficient to form a hydrophilic surface coating on the silicone ophthalmic device by van der Waals dispersion forces.

In non-limiting illustrative embodiments, the silicone ophthalmic device can be released from a mold assembly and then contacted with an aqueous packaging solution containing the copolymer disclosed herein. For example, the silicone ophthalmic device can be transferred to an individual lens package containing a buffered saline solution containing at least the copolymer disclosed herein and subjected to sterilization by, e.g., autoclaving.

In some embodiments, a suitable temperature can range from about 100° C. to about 130° C. In some embodiments, a suitable time period can range from about 10 minutes to about 1.5 hours.

While not wishing to be bound by theory, it is believed that when heating the aqueous solution to the temperature sufficient to form a hydrophilic surface coating on the silicone ophthalmic device by van der Waals dispersion forces, the temperature of the surface of the silicone ophthalmic device will be significantly above the glass transition temperature (Tg) of moieties at its surface. In addition, the copolymer will undergo a crosslinking reaction in which given ones of the ring-opening reactive functionalities of the monomeric units derived from an ethylenically unsaturated-containing monomer having ring-opening reactive functionalities of the copolymer will crosslink with other given ones of the ring-opening reactive functionalities of the monomeric units derived from an ethylenically unsaturated-containing monomer having ring-opening reactive functionalities of the copolymer thereby forming a hydrophobic nano-gel. While the temperature of the surface of the silicone ophthalmic device is significantly above the glass transition temperature (Tg) of moieties at its surface, the hydrophobic nano-gel can adhere to the surface of the silicone ophthalmic device. Once the aqueous solution is allowed to cool, the hydrophobic nano-gel in the aqueous solution becomes hydrophilic and stabilizes on the surface of the silicone ophthalmic device thereby forming a surface modified silicone ophthalmic device. Therefore, the cooled down hydrophilic nano-gel can hydrophilize and lubricate the surface of the silicone ophthalmic device with a hydrophilic surface coating.

In non-limiting illustrative embodiments, the copolymer disclosed herein is present in the aqueous packaging solution in an amount ranging from about 0.01 wt. % to about 2 wt. %, based on the total weight of the aqueous packaging solution. In some embodiments, the copolymer disclosed herein is present in the aqueous packaging solution in an amount ranging from about 0.1 wt. % to about 0.5 wt. %, based on the total weigh of the aqueous packaging solution.

Appropriate packaging designs and materials are known in the art. A plastic package is releasably sealed with a film. Suitable sealing films are known in the art and include foils, polymer films and mixtures thereof. The sealed packages containing the lenses are then sterilized to ensure a sterile product. Suitable sterilization means and conditions are known in the art and include, for example, steam sterilizing or autoclaving of the sealed container at temperatures of about 120° C. or higher.

The aqueous packaging solutions of the illustrative embodiments are physiologically compatible. Specifically, the aqueous packaging solution must be “ophthalmically safe” for use with a lens such as a contact lens, meaning that a contact lens treated with the solution is generally suitable and safe for direct placement on the eye without rinsing, that is, the aqueous packaging solution is safe and comfortable for daily contact with the eye via a contact lens that has been wetted with the solution. An ophthalmically safe solution has a tonicity and pH that is compatible with the eye and includes materials, and amounts thereof, that are non-cytotoxic according to ISO standards and U.S. Food & Drug Administration (FDA) regulations.

The aqueous packaging solution should also be sterile in that the absence of microbial contaminants in the product prior to release must be statistically demonstrated to the degree necessary for such products. The liquid media suitable in the present invention are selected to have no substantial detrimental effect on the lens being treated or cared for and to allow or even facilitate the present lens treatment or treatments. The liquid media are preferably aqueous-based. A particularly suitable aqueous liquid medium is that derived from saline, for example, a conventional saline solution or a conventional buffered saline solution.

The pH of the aqueous packaging solutions is maintained within the range of about 6 to about 9, and preferably about 6.5 to about 7.8. As mentioned above, additional buffer may optionally be added, such as boric acid, sodium borate, potassium citrate, sodium citrate, citric acid, sodium bicarbonate, various mixed phosphate buffers (including combinations of Na₂HPO₄, NaH₂PO₄ and KH₂PO₄), hydrates thereof and the like and mixtures thereof. Generally, buffers will be used in amounts ranging from about 0.05 to about 2.5 percent by weight, and preferably from about 0.1 to about 1.5 percent by weight of the solution. However, according to certain embodiments, tris(hydroxymethyl)aminomethane, or salts thereof, function as the sole buffer.

In some embodiments, the aqueous packaging solution can further comprise one or more buffer agents. Suitable one or more buffer agents include, for example, phosphate buffer agents, borate buffer agents, citrate buffer agents, and the like. A suitable phosphate buffer agent can be any known phosphate buffer agents. In some embodiments, the phosphate buffer agent comprises one or more of sodium hydrogen phosphate monobasic, sodium hydrogen phosphate dibasic, potassium hydrogen phosphate monobasic and potassium hydrogen phosphate dibasic and any suitable hydrate thereof, e.g., monohydrate and heptahydrate. A suitable borate buffer agent can be any known borate buffer agents. In some embodiments, the borate buffer agent comprises one or more of boric acid and sodium borate. A suitable citrate buffer agent can be any known citrate buffer agents. In some embodiments, the citrate buffer agent comprises one or more of citric acid and sodium citrate.

In some embodiments, the one or more buffer agents are present in the aqueous packaging solution in an amount ranging from about 0.001 wt. % to about 2 wt. %, based on the total weight of the packaging solution. In some embodiments, the phosphate buffer agent is present in the packaging solution in an amount ranging from about 0.001 wt. % to about 1 wt. %, based on the total weight of the packaging solution.

Typically, the aqueous packaging solutions are also adjusted with tonicity agents, to approximate the osmotic pressure of normal lacrimal fluids which is equivalent to a 0.9 percent solution of sodium chloride or 2.5 percent of glycerol solution. The solutions are made substantially isotonic with physiological saline used alone or in combination, otherwise if simply blended with sterile water and made hypotonic or made hypertonic the lenses will lose their desirable optical parameters. Correspondingly, excess saline may result in the formation of a hypertonic solution which will cause stinging and eye irritation.

Examples of suitable tonicity adjusting agents include, but are not limited to, sodium and potassium chloride, dextrose, glycerin, calcium and magnesium chloride and the like and mixtures thereof. These agents are typically used individually in amounts ranging from about 0.01% w/v to about 2.5% w/v and preferably from about 0.2% w/v to about 1.5% w/v. Preferably, the tonicity agent will be employed in an amount to provide a final osmotic value of at least about 200 mOsm/kg, or from about 200 mOsm/kg to about 400 mOsm/kg, or from about 250 mOsm/kg to about 350 mOsm/kg, or from about 280 mOsm/kg to about 320 mOsm/kg.

If desired, one or more additional components can be included in the packaging solution. Such an additional component or components are chosen to impart or provide at least one beneficial or desired property to the packaging solution. Such additional components may be selected from components which are conventionally used in one or more ophthalmic device care compositions. Examples of such additional components include, but are not limited to, cleaning agents, wetting agents, nutrient agents, sequestering agents, viscosity builders, contact lens conditioning agents, antioxidants, and the like and mixtures thereof. These additional components may each be included in the packaging solutions in an amount effective to impart or provide the beneficial or desired property to the packaging solutions. For example, such additional components may be included in the packaging solutions in amounts similar to the amounts of such components used in other, e.g., conventional, contact lens care products.

Suitable sequestering agents include, but are not limited to, disodium ethylene diamine tetraacetate, alkali metal hexametaphosphate, citric acid, sodium citrate and the like and mixtures thereof.

Suitable viscosity builders include, but are not limited to, hydroxyethyl cellulose, hydroxymethyl cellulose, polyvinyl pyrrolidone, polyvinyl alcohol and the like and mixtures thereof.

Suitable antioxidants include, but are not limited to, sodium metabisulfite, sodium thiosulfate, N-acetylcysteine, butylated hydroxyanisole, butylated hydroxytoluene and the like and mixtures thereof.

The method of packaging and storing a silicon ophthalmic device such as a contact lens includes at least packaging a silicone ophthalmic device immersed in the aqueous packaging solution containing the copolymer disclosed herein and sterilizing the packaged solution. The method may include immersing the ophthalmic device in the aqueous packaging solution containing the copolymer disclosed herein prior to delivery to the customer/wearer, directly following manufacture of the contact lens. Alternately, the packaging and storing in the aqueous packaging solution containing the copolymer disclosed herein may occur at an intermediate point before delivery to the ultimate customer (wearer) but following manufacture and transportation of a contact lens in a dry state, wherein a dry contact lens is hydrated by immersing the contact lens in the aqueous packaging solution containing the copolymer disclosed herein. Consequently, a package for delivery to a customer may include a sealed container containing one or more unused surface modified contact lenses immersed in the aqueous packaging solution.

After polymerization is completed, any non-covalently bonded monomers, oligomers or polymers formed can be removed, for example, by treatment with a suitable solvent. The resulting surface modified ophthalmic device can then be used “as is”. In other words, no additional surface treatment steps will have to be carried out to modify the resulting surface modified ophthalmic device. As used herein, the phrase “without any additional surface treatment steps” shall be understood to mean that the exterior surface of the surface modified ophthalmic device of the illustrative embodiments is not further treated to modify the surface thereof by, for example, oxidation treatments, plasma treatments, grafting treatments, coating treatments and the like. However, it shall be understood that coatings such as color or other cosmetic enhancement may be applied to devices disclosed herein.

The following examples are provided to enable one skilled in the art to practice the invention and are merely illustrative of the invention. The examples should not be read as limiting the scope of the invention as defined in the claims.

In the examples, the following abbreviations are used.
NVP: N-vinyl-2-pyrrolidone.
DMA: N,N-dimethylacrylamide.
EGDMA: Ethylene glycol dimethacrylate.
MMA: Methyl methacrylate.
MEMA: 2-Methoxyethyl methacrylate or Ethylene glycol methyl ether methacrylate.
EGDMA: Ethylene glycol dimethacrylate.
IMVT: 1,4-bis(4-(2-methacryloxyethyl)phenylamino)anthraquinone.
Vazo™ 64: azo bis-isobutylnitrile (AIBN).

SA monomer: A compound having the structure:

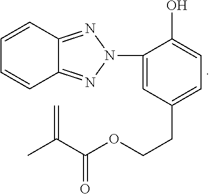 [SMILES: C=C(C)C(=O)OCCc1ccc(O)c(-n2nc3ccccc3n2)c1]

X-22-1666C: A compound available from ShinEtsu and having the following structure:

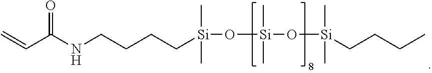

### Contact Angle

Captive bubble contact angle data was collected on a First Ten Angstroms FTA-1000 prop Shape Instrument. All samples were rinsed in HPLC grade water prior to analysis in order to remove components of the packaging solution from the sample surface. Prior to data collection the surface tension of the water used for all experiments was measured using the pendant drop method. In order for the water to qualify as appropriate for use, a surface tension value of 70-72 dynes/cm was expected. All lens samples were placed onto a curved sample holder and submerged into a quartz cell filled with HPLC grade water. Advancing and receding captive bubble contact angles were collected for each sample. The advancing contact angle is defined as the angle measured in water as the air bubble is retracting from the lens surface (water is advancing across the surface). All captive bubble data was collected using a high-speed digital camera focused onto the sample/air bubble interface. The contact angle was calculated at the digital frame just prior to contact line movement across the sample/air bubble interface. The receding contact angle is defined as the angle measured in water as the air bubble is expanding across the sample surface (water is receding from the surface).

Sessile Drop Method—Contact angles reported in the Examples were also determined according to the Sessile Drop Method first developed by Zisman, W. A., et al., J. Colloid Sci., Vol. 1, p. 513 (1946). A plastic film with support was placed on a flat plate in a Rane-Hart goniometer. A drop of liquid of interest (distilled water, buffered saline or any other liquid of interest) was applied to the film through a metered syringe. The angle was read from the viewer, after adjusting the baseline.

### Example 1

### Preparation of Poly(GMA-DMA).

Into a 150 mL round bottom flask with a stir bar was added AIBN (8.7 mg, 0.053 mmol), dimethylacrylamide (9.4 g, 95 mmol), and glycidyl methacrylate (1.5 g, 10.6 mmol) into anhydrous acetonitrile (50 mL). The flask was purged with nitrogen for 30 minutes and then transferred to a pre-heat oil bath at 65° C. The solution was stirred and heated overnight.

The solution was cooled to room temperature and then precipitate into diethyl ether (400 mL) to form a white solid product. The white solid product was then redissolved in acetonitrile (50 mL) then precipitated in diethyl ether two more times to form a white solid product poly (GMA-DMA) having 10 mol % of GMA and 90 mol % of DMA (8.5 g, yield=78%).

### Examples 2-4

### Preparation of Surface-Modified Silicone Contact Lens.

The silicone contact lenses were prepared using the reaction components in the monomeric mixtures listed in Table 1 below, as amounts per weight percent.

TABLE 1

| | Ex. 2 | Ex. 3 | Ex. 4 |
|---|---|---|---|
| Formulation | | | |
| X22-1666C | 40 | 45 | 45 |
| MMA | 5 | 5 | 5 |
| MEMA | 9 | 6 | 9 |
| EDGMA | 0.5 | 0.4 | 0.3 |
| DMA | 10 | 13 | 17 |
| NVP | 32 | 27 | 20 |
| AIBN | 0.4 | 0.4 | 0.4 |
| SA | 3 | 3 | 3 |
| IMVT | 0.02 | 0.2 | 0.2 |
| Total | 99.8 | 100.0 | 99.9 |
| Properties | | | |
| Modulus (gf/mm²) | 69 ± 3 | 53 ± 1 | 54 ± 2 |
| Water Content (%) | 47 | 47 | 44.5 |
| Dk (Barrer) | 72 | 78 | 78 |

The monomeric mixtures were cast on the anterior and posterior surfaces of polypropylene molds. The molds were thermally cured at 100° C. for 1 hour. The cured lenses were dry-released and hydrated in DI water for 10 minutes.

A polymer coating solution was prepared by dissolving the poly(GMA-DMA) of Example 1 in water at a concentration of 3 mg/mL in a phosphate buffered solution. The silicone contact lenses of Examples 2-4 were individually placed in a polypropylene blister with 1 mL of the polymer coating solution and sealed with aluminum foil. The sealed polypropylene blisters were sterilized for 20 minutes by autoclaving at 120° C. for 20 minutes, then allowed to cool to room temperature. The whole autoclave process took around 1.5 hours.

Contact angles were measured for both coated and uncoated lenes multiple times as presented below in Tables 2.

TABLE 2
Sessile Drop Contact Angle

| | Uncoated Lens | Coated Lens | Coated Lens |
|---|---|---|---|
| Ex. | (Anterior) | (Anterior) | (Posterior) |
| 2 | 75 | 40 | 42 |
| 3 | 75 | 40 | 41 |
| 4 | 73 | 39 | 41 |

As can be seen, the consistent contact angle on both the anterior and posterior surfaces shows that the coated lenses had a uniform coating on both surfaces.

According to an aspect of the present disclosure, a surface modified silicone ophthalmic device comprises a silicone ophthalmic device having a hydrophilic surface coating attached to a surface of the silicone ophthalmic device by van der Waals dispersion forces, the hydrophilic surface coating comprising a copolymer comprising (i) monomeric units derived from an ethylenically unsaturated-containing monomer having ring-opening reactive functionalities, and (ii) monomeric units derived from a hydrophilic monomer having an ethylenically unsaturated reactive group.

In non-limiting illustrative embodiments, as may be combined with one or more of the preceding paragraphs, the silicone ophthalmic device is derived from a polymerization product of a monomeric mixture comprising from about 30 wt. % to about 90 wt. %, based on the total weight of the monomeric mixture, of one or more ophthalmic device-forming silicon monomers.

In non-limiting illustrative embodiments, as may be combined with one or more of the preceding paragraphs, the silicone ophthalmic device is derived from a polymerization product of a monomeric mixture comprising from about 30 wt. % to about 70 wt. %, based on the total weight of the monomeric mixture, of one or more ophthalmic device-forming silicon monomers.

In non-limiting illustrative embodiments, as may be combined with one or more of the preceding paragraphs, the ethylenically unsaturated-containing monomer having ring-opening reactive functionalities comprises 2 to about 18 carbon atoms which is substituted by an epoxy group.

In non-limiting illustrative embodiments, as may be combined with one or more of the preceding paragraphs, the hydrophilic monomer is selected from the group consisting of an unsaturated carboxylic acid, an acrylamide, a vinyl lactam, a hydroxyl-containing-(meth)acrylate, a hydrophilic vinyl carbonate, a hydrophilic vinyl carbamate monomer, a hydrophilic oxazolone monomer, vinyl chloroformate and mixtures thereof.

In non-limiting illustrative embodiments, as may be combined with one or more of the preceding paragraphs, the hydrophilic monomer is selected from the group consisting of an unsaturated carboxylic acid, an acrylamide, a vinyl lactam, a hydroxyl-containing-(meth)acrylate and mixtures thereof.

In non-limiting illustrative embodiments, as may be combined with one or more of the preceding paragraphs, the ethylenically unsaturated-containing monomer having ring-opening reactive functionalities is glycidyl methacrylate and the hydrophilic monomer is dimethylacrylamide.

In non-limiting illustrative embodiments, as may be combined with one or more of the preceding paragraphs, the copolymer comprises from about 5 mol % to about 25 mol % of the monomeric units derived from the ethylenically unsaturated-containing monomer having ring-opening reactive functionalities and from about 75 mol % to about 95 mol % of the monomeric units derived from the hydrophilic monomer.

In non-limiting illustrative embodiments, as may be combined with one or more of the preceding paragraphs, the copolymer comprises from about 10 mol % to about 25 mol % of the monomeric units derived from the ethylenically unsaturated-containing monomer having ring-opening reactive functionalities and from about 75 mol % to about 90 mol % of the monomeric units derived from the hydrophilic monomer.

In non-limiting illustrative embodiments, as may be combined with one or more of the preceding paragraphs, the copolymer is a brush copolymer.

In non-limiting illustrative embodiments, as may be combined with one or more of the preceding paragraphs, the copolymer is a random copolymer.

In non-limiting illustrative embodiments, as may be combined with one or more of the preceding paragraphs, the hydrophilic surface coating has a uniform thickness on an anterior surface of the silicone ophthalmic device and on a posterior surface of the silicone ophthalmic device.

In non-limiting illustrative embodiments, as may be combined with one or more of the preceding paragraphs, the hydrophilic surface coating has a thickness of about 0.1 μm to about 2 μm on an anterior surface of the silicone ophthalmic device and a thickness of about 0.1 μm to about 2 μm on a posterior surface of the silicone ophthalmic device.

In non-limiting illustrative embodiments, as may be combined with one or more of the preceding paragraphs, the silicone ophthalmic device is a silicone ophthalmic lens.

In non-limiting illustrative embodiments, as may be combined with one or more of the preceding paragraphs, the silicone ophthalmic lens is a silicone contact lens or a silicone intraocular lens.

In non-limiting illustrative embodiments, as may be combined with one or more of the preceding paragraphs, the silicone ophthalmic device is a silicone hydrogel continuous-wear lens.

According to another aspect of the present disclosure, a method for making a surface modified silicone ophthalmic device comprises attaching a hydrophilic surface coating to a surface of a silicone ophthalmic device by van der Waals dispersion forces, the hydrophilic surface coating comprising a copolymer comprising (i) monomeric units derived from an ethylenically unsaturated-containing monomer having ring-opening reactive functionalities, and (ii) monomeric units derived from a hydrophilic monomer having an ethylenically unsaturated reactive group.

In non-limiting illustrative embodiments, as may be combined with one or more of the preceding paragraphs, attaching the hydrophilic surface coating to the surface of the silicone ophthalmic device comprises immersing the silicone ophthalmic device into a solution comprising the copolymer comprising (i) monomeric units derived from an ethylenically unsaturated-containing monomer having ring-opening reactive functionalities, and (ii) monomeric units derived from a hydrophilic monomer having an ethylenically unsaturated reactive group, and heating the silicone ophthalmic device in the solution to a temperature and for a time period sufficient to form the hydrophilic surface coating

In non-limiting illustrative embodiments, as may be combined with one or more of the preceding paragraphs, attaching the hydrophilic surface coating to the surface of the silicone ophthalmic device comprises:
immersing the silicone ophthalmic device in an aqueous packaging solution comprising the copolymer comprising (i) monomeric units derived from an ethylenically unsaturated-containing monomer having ring-opening reactive functionalities, and (ii) monomeric units derived from a hydrophilic monomer having an ethylenically unsaturated reactive group, wherein the aqueous packaging solution has an osmolality of at least about 150 mOsm/kg and a pH from about 6 to about 9,
packaging the aqueous packaging solution and the silicone ophthalmic device in a manner preventing contamination of the silicone ophthalmic device by microorganisms, and
sterilizing the packaged aqueous packaging solution and the silicone ophthalmic device.

In non-limiting illustrative embodiments, as may be combined with one or more of the preceding paragraphs, sterilizing the packaged aqueous packaging solution and the silicone ophthalmic device comprises autoclaving the packaged aqueous packaging solution and the silicone ophthalmic device.

In non-limiting illustrative embodiments, as may be combined with one or more of the preceding paragraphs, the silicone ophthalmic device is derived from a polymerization product of a monomeric mixture comprising from about 30 wt. % to about 90 wt. %, based on the total weight of the monomeric mixture, of one or more ophthalmic device-forming silicon monomers.

In non-limiting illustrative embodiments, as may be combined with one or more of the preceding paragraphs, the silicone ophthalmic device is derived from a polymerization product of a monomeric mixture comprising from about 30 wt. % to about 70 wt. %, based on the total weight of the monomeric mixture, of one or more ophthalmic device-forming silicon monomers.

In non-limiting illustrative embodiments, as may be combined with one or more of the preceding paragraphs, the ethylenically unsaturated-containing monomer having ring-opening reactive functionalities comprises 2 to about 18 carbon atoms which is substituted by an epoxy group.

In non-limiting illustrative embodiments, as may be combined with one or more of the preceding paragraphs, the hydrophilic monomer is selected from the group consisting of an unsaturated carboxylic acid, an acrylamide, a vinyl lactam, a hydroxyl-containing-(meth)acrylate, a hydrophilic vinyl carbonate, a hydrophilic vinyl carbamate monomer, a hydrophilic oxazolone monomer, vinyl chloroformate and mixtures thereof.

In non-limiting illustrative embodiments, as may be combined with one or more of the preceding paragraphs, the hydrophilic monomer is selected from the group consisting of an unsaturated carboxylic acid, an acrylamide, a vinyl lactam, a hydroxyl-containing-(meth)acrylate and mixtures thereof.

In non-limiting illustrative embodiments, as may be combined with one or more of the preceding paragraphs, the ethylenically unsaturated-containing monomer having ring-opening reactive functionalities is glycidyl methacrylate and the hydrophilic monomer is dimethylacrylamide.

In non-limiting illustrative embodiments, as may be combined with one or more of the preceding paragraphs, the copolymer comprises from about 5 mol % to about 25 mol % of the monomeric units derived from the ethylenically unsaturated-containing monomer having ring-opening reactive functionalities and from about 75 mol % to about 95 mol % of the monomeric units derived from the hydrophilic monomer.

In non-limiting illustrative embodiments, as may be combined with one or more of the preceding paragraphs, the copolymer comprises from about 10 mol % to about 25 mol % of the monomeric units derived from the ethylenically unsaturated-containing monomer having ring-opening reactive functionalities and from about 75 mol % to about 90 mol % of the monomeric units derived from the hydrophilic monomer.

In non-limiting illustrative embodiments, as may be combined with one or more of the preceding paragraphs, the copolymer is a brush copolymer.

In non-limiting illustrative embodiments, as may be combined with one or more of the preceding paragraphs, the copolymer is a random copolymer.

In non-limiting illustrative embodiments, as may be combined with one or more of the preceding paragraphs, the hydrophilic surface coating has a uniform thickness on an anterior surface of the silicone ophthalmic device and on a posterior surface of the silicone ophthalmic device.

In non-limiting illustrative embodiments, as may be combined with one or more of the preceding paragraphs, the hydrophilic surface coating has a thickness of about 0.1 μm to about 2 μm on an anterior surface of the silicone ophthalmic device and a thickness of about 0.1 μm to about 2 μm on a posterior surface of the silicone ophthalmic device.

In non-limiting illustrative embodiments, as may be combined with one or more of the preceding paragraphs, the silicone ophthalmic device is a silicone ophthalmic lens.

In non-limiting illustrative embodiments, as may be combined with one or more of the preceding paragraphs, the silicone ophthalmic lens is a silicone contact lens or a silicone intraocular lens.

In non-limiting illustrative embodiments, as may be combined with one or more of the preceding paragraphs, the silicone ophthalmic device is a silicone hydrogel continuous-wear lens.

Various features disclosed herein are, for brevity, described in the context of a single embodiment, but may also be provided separately or in any suitable sub-combination. All combinations of the embodiments are specifically embraced by the illustrative embodiments disclosed herein just as if each and every combination was individually and explicitly disclosed. In addition, all sub-combinations listed in the embodiments describing such variables are also specifically embraced by the present compositions and are disclosed herein just as if each and every such sub-combination was individually and explicitly disclosed herein.

It will be understood that various modifications may be made to the embodiments disclosed herein. Therefore, the above description should not be construed as limiting, but merely as exemplifications of preferred embodiments. For example, the functions described above and implemented as the best mode for operating the present invention are for illustration purposes only. Other arrangements and methods may be implemented by those skilled in the art without departing from the scope and spirit of this invention. Moreover, those skilled in the art will envision other modifications within the scope and spirit of the features and advantages appended hereto.

What is claimed is:

1. A surface modified silicone ophthalmic device comprising a silicone ophthalmic device having a hydrophilic surface coating attached to a surface of the silicone ophthalmic device by van der Waals dispersion forces, the hydrophilic surface coating comprising a copolymer comprising (i) monomeric units derived from an ethylenically unsaturated-containing monomer having ring-opening reactive functionalities, and (ii) monomeric units derived from a hydrophilic monomer having an ethylenically unsaturated reactive group.

2. The surface modified silicone ophthalmic device according to claim 1, wherein the silicone ophthalmic device is derived from a polymerization product of a monomeric mixture comprising from about 30 wt. % to about 90 wt. %, based on the total weight of the monomeric mixture, of one or more ophthalmic device-forming silicon monomers.

3. The surface modified silicone ophthalmic device according to claim 1, wherein the ethylenically unsaturated-containing monomer having ring-opening reactive functionalities comprises 2 to about 18 carbon atoms which is substituted by an epoxy group.

4. The surface modified silicone ophthalmic device according to claim 3, wherein the hydrophilic monomer is selected from the group consisting of an unsaturated carboxylic acid, an acrylamide, a vinyl lactam, a hydroxyl-containing-(meth)acrylate and mixtures thereof.

5. The surface modified silicone ophthalmic device according to claim 1, wherein the ethylenically unsaturated-containing monomer having ring-opening reactive functionalities is glycidyl methacrylate and the hydrophilic monomer is dimethylacrylamide.

6. The surface modified silicone ophthalmic device according to claim 1, wherein the copolymer comprises from about 5 mol % to about 25 mol % of the monomeric units derived from the ethylenically unsaturated-containing monomer having ring-opening reactive functionalities and from about 75 mol % to about 95 mol % of the monomeric units derived from the hydrophilic monomer.

7. The surface modified silicone ophthalmic device according to claim 5, wherein the copolymer comprises from about 10 mol % to about 25 mol % of the monomeric units derived from the ethylenically unsaturated-containing monomer having ring-opening reactive functionalities and from about 75 mol % to about 90 mol % of the monomeric units derived from the hydrophilic monomer.

8. The surface modified silicone ophthalmic device according to claim 1, wherein the copolymer is a brush copolymer.

9. The surface modified silicone ophthalmic device according to claim 1, wherein the hydrophilic surface coating has a uniform thickness on an anterior surface of the silicone ophthalmic device and on a posterior surface of the silicone ophthalmic device.

10. The surface modified silicone ophthalmic device according to claim 1, which is a surface modified silicone contact lens.

11. The surface modified silicone ophthalmic device according to claim 1, which is a surface modified silicone hydrogel continuous-wear lens.

12. A method for making a surface modified silicone ophthalmic device comprising attaching a hydrophilic surface coating to a surface of a silicone ophthalmic device by van der Waals dispersion forces, the hydrophilic surface coating comprising a copolymer comprising (i) monomeric units derived from an ethylenically unsaturated-containing monomer having ring-opening reactive functionalities, and (ii) monomeric units derived from a hydrophilic monomer having an ethylenically unsaturated reactive group.

13. The method according to claim 12, wherein attaching the hydrophilic surface coating to the surface of the silicone ophthalmic device comprises immersing the silicone ophthalmic device into a solution comprising the copolymer comprising (i) monomeric units derived from an ethylenically unsaturated-containing monomer having ring-opening reactive functionalities, and (ii) monomeric units derived from a hydrophilic monomer having an ethylenically unsaturated reactive group, and heating the silicone ophthalmic device in the solution to a temperature and for a time period sufficient to form the hydrophilic surface coating.

14. The method according to claim 12, wherein attaching the hydrophilic surface coating to the surface of the silicone ophthalmic device comprises:
immersing the silicone ophthalmic device in an aqueous packaging solution comprising the copolymer comprising (i) monomeric units derived from an ethylenically unsaturated-containing monomer having ring-opening reactive functionalities, and (ii) monomeric units derived from a hydrophilic monomer having an ethylenically unsaturated reactive group, wherein the aqueous packaging solution has an osmolality of at least about 150 mOsm/kg and a pH from about 6 to about 9;
packaging the aqueous packaging solution and the silicone ophthalmic device in a manner preventing contamination of the silicone ophthalmic device by microorganisms; and
sterilizing the packaged aqueous packaging solution and the silicone ophthalmic device.

15. The method according to claim 14, wherein sterilizing the packaged aqueous packaging solution and the silicone ophthalmic device comprises autoclaving the packaged aqueous packaging solution and the silicone ophthalmic device.

16. The method according to claim 12, wherein the ethylenically unsaturated-containing monomer having ring-opening reactive functionalities comprises 2 to about 18 carbon atoms which is substituted by an epoxy group, and the hydrophilic monomer having an ethylenically unsaturated reactive group is selected from the group consisting of an unsaturated carboxylic acid, an acrylamide, a vinyl lactam, a hydroxyl-containing-(meth)acrylate and mixtures thereof.

17. The method according to claim 12, wherein the ethylenically unsaturated-containing monomer having ring-opening reactive functionalities is glycidyl methacrylate and the hydrophilic monomer having an ethylenically unsaturated reactive group is dimethylacrylamide.

18. The method according to claim 12, wherein the copolymer comprises from about 5 mol % to about 25 mol % of the monomeric units derived from the ethylenically unsaturated-containing monomer having ring-opening reactive functionalities and from about 75 mol % to about 95 mol % of the monomeric units derived from the hydrophilic monomer.

19. The method according to claim 12, wherein the hydrophilic surface coating has a uniform thickness on an anterior surface of the silicone ophthalmic device and on a posterior surface of the silicone ophthalmic device.

20. The method according to claim 12, wherein the surface modified silicone ophthalmic device is a surface modified silicone hydrogel continuous-wear lens.
